(19) United States
(12) Patent Application Publication
(10) Pub. No.: US 2025/0069288 A1
(43) Pub. Date: Feb. 27, 2025

(54) SYSTEMS AND METHODS FOR AUTOMATED MESH CLEANUP

(71) Applicant: SDC U.S. SmilePay SPV, Nashvill, TN (US)

(72) Inventors: Ryan Amelon, Nashville, TN (US); Alex Powers, Nashville, TN (US); Tim Wucher, Windhoek (NA)

(73) Assignee: SDC U.S. SmilePay SPV, Nashville, TN (US)

(21) Appl. No.: 18/236,810

(22) Filed: Aug. 22, 2023

(51) Int. Cl. G06T 11/00; A61C 7/00; A61C 9/00; G06T 5/00; G16H 30/40

(52) U.S. Cl. CPC G06T 11/003; A61C 7/002; A61C 9/0053; G06T 5/00; G16H 30/40; G06T 2200/04; G06T 2207/10016; G06T 2207/20081; G06T 2207/20182; G06T 2207/30036

(57) ABSTRACT

Disclosed is a computer-implemented method that includes receiving, by one or more processors, a three-dimensional (3D) representation of a user's mouth where the 3D representation is a mesh representation comprising a plurality of surfaces; mapping, by the one or more processors, the 3D representation of the user's mouth into a two-dimensional (2D) space; encoding, one or more 3D surface characteristics of the plurality of surfaces using one or more channels of a 2D representation; applying, by the one or more processors, a machine learning model to the representation of the user's mouth in 2D space, the machine learning model trained to enhance the representation of the user's mouth in 2D space; and mapping, by the one or more processors, the enhanced representation of the user's mouth in 2D space to an enhanced representation of the user's mouth in 3D space using the one or more surface characteristics.

### TECHNICAL FIELD

The present disclosure relates generally to the field of dental treatment, and more specifically to systems and methods for mesh cleanup of captured digital representations of a user's mouth.

### BACKGROUND

Digital representations of a person's teeth can be utilized for purposes of dental or orthodontic care. For example, three dimensional (3D) representations of teeth may be used to manufacture dental aligners, generate treatment plans, track teeth movement, etc. One example 3D representation of a person's teeth may be a mesh representing the person's teeth. Conventionally, mesh cleanup and sculpting are manually performed in software by trained specialists. However, the manual process is time-consuming, subjective, and inconsistent across specialists. Further, such digital representations of teeth may include artifacts, holes, or other imperfections as compared to the person's actual teeth. The artifacts, holes, or other imperfections could impede generating a sufficient 3D representation needed for dental or orthodontic care.

### SUMMARY

In one aspect, this disclosure is directed to a system. The system includes a processor and a non-transitory computer readable medium containing instructions that when executed by the processor causes the processor to perform operations comprising receiving a three-dimensional (3D) representation of a user's mouth where the 3D representation is a mesh representation comprising a plurality of surfaces, mapping the 3D representation of the user's mouth into a two-dimensional (2D) space, encoding one or more 3D surface characteristics of the plurality of surfaces using one or more channels of a 2D representation, determining an enhanced representation of the user's mouth in 2D space by executing a machine learning model on the representation of the user's mouth in 2D space, mapping the enhanced representation of the user's mouth in 2D space to an enhanced 3D representation of the user's mouth using the encoded one or more 3D surface characteristics of the plurality of surfaces, and outputting the enhanced 3D representation.

In another aspect, this disclosure is directed to a method. The method includes receiving, by one or more processors, a three-dimensional (3D) representation of a user's mouth where the 3D representation is a mesh representation comprising a plurality of surfaces; mapping, by the one or more processors, the 3D representation of the user's mouth into a two-dimensional (2D) space; encoding, by the one or more processors, one or more 3D surface characteristics of the plurality of surfaces using one or more channels of a 2D representation; applying, by the one or more processors, a machine learning model to the representation of the user's mouth in 2D space, the machine learning model trained to enhance the representation of the user's mouth in 2D space; and mapping, by the one or more processors, the enhanced representation of the user's mouth in 2D space to an enhanced representation of the user's mouth in 3D space using the encoded one or more 3D surface characteristics.

In yet another aspect, this disclosure is directed to a method. The method includes receiving, by one or more processors, a two-dimensional (2D) representation of a user's mouth; converting, by the one or more processors, the 2D representation into a three-dimensional (3D) representation of the user's mouth, wherein the 3D representation is a mesh representation comprising a plurality of surfaces; mapping, by the one or more processors, the 3D representation of the user's mouth into a two-dimensional (2D) space; encoding, by the one or more processors, one or more 3D surface characteristics of the plurality of surfaces using one or more channels of the 2D representation; applying, by the one or more processors, a machine learning model to the representation of the user's mouth in 2D space, the machine learning model trained to enhance the representation of the user's mouth in 2D space; and mapping, by the one or more processors, the enhanced representation of the user's mouth in 2D space to an enhanced representation of the user's mouth in 3D space using the encoded one or more 3D surface characteristics.

Various other embodiments and aspects of the disclosure will become apparent based on the drawings and detailed description of the following disclosure.

### BRIEF DESCRIPTION OF THE DRAWINGS

FIG. 1 is a block diagram of a computer-implemented system including a treatment planning computing system including a mesh cleanup application utilizing a machine learning architecture, according to an illustrative embodiment.

FIG. 2 is an illustration of a user using a mirror to capture an image with a rear-facing camera on a user device, according to an illustrative embodiment.

FIG. 3 is a block diagram of an example system using supervised learning to clean and sculpt meshes, according to an illustrative embodiment.

FIG. 4 is a block diagram of an example system including the treatment planning computing system of FIG. 1 configured to clean a digital representation of a user's mouth for a downstream application, according to an illustrative embodiment.

FIGS. 5A-5E are flow diagrams of the mesh cleanup application of FIG. 1, according to illustrative embodiments.

FIG. 6 is an illustration of a digital model of a user's teeth, according to an illustrative embodiment.

FIG. 7 is an illustration of surface characteristics of a surface of the digital model of FIG. 6, according to an illustrative embodiment.

FIG. 8 is another flow diagram of the mesh cleanup application of FIG. 1, according to an illustrative embodiment.

### DETAILED DESCRIPTION

The present disclosure is directed to systems and methods for automating a mesh cleanup process specific to 3D teeth meshes for the purpose of dental or orthodontic care, including aligner therapy to reposition one or more teeth of a user. Given a digital representation (e.g., a 2D or 3D representation) of a user's mouth, a mesh cleanup application cleans a 3D mesh representation of the user's mouth. The mesh representation is a representation of polygons each having surfaces in x, y, and z-directions. For ease of description, the surfaces of each of the polygons in an orientation in space is described as the mesh representation. As described herein, cleaning (or otherwise enhancing) the mesh representation of the user's mouth may include filling in holes/gaps, removing artifacts (e.g., replicated polygons), smoothing polygons, and other operations configured to improve the detail of one or more dental features in a digital representation. The mesh cleanup application automatically sculpts the polygons in space such that the cleaned mesh representation is a more accurate representation of the true teeth and gums of which the digital representation is based on.

The systems and method described herein may have many benefits over existing computing systems or manual systems for enhancing a mesh representation. For example, the mesh cleanup application allows for immediate (or near immediate) mesh (or surface, or polygon) cleanup, as well as standardization of mesh cleanup that is not influenced by the subjective nature of manual processes. The automated mesh cleanup and processing improves the processing speed, repeatability, and consistency of performing such mesh clean up processes. Standardizing the mesh cleanup process reduces/limits computational resources consumed by a system that would otherwise be subjected to multiple iterations of cleaning meshes. The systems and methods described herein further improve upon existing computing systems or manual systems for enhancing meshes by utilizing specific rules and techniques not taught in the prior art or capable of being performed by a human, such as mapping a 3D representation of teeth into a 2D space, encoding one or more surfaces (e.g., 3D representations) of the mesh representation using one or more channels of the 2D representation, determining a clean (enhanced) representation of the teeth in 2D space by executing a machine learning model on the representation of the user's mouth in 2D space, and mapping the clean (enhanced) representation of the user's mouth in 2D space to a clean (enhanced) 3D representation of the user's mouth.

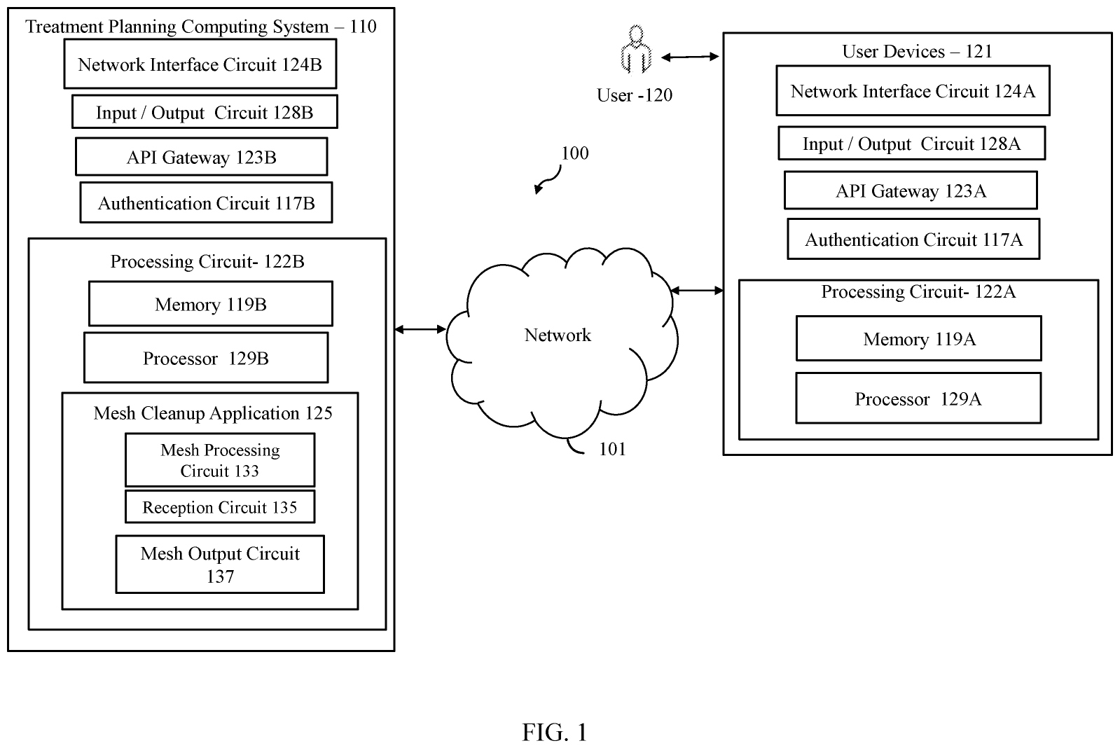

Referring now to FIG. 1, a block diagram of a computer-implemented system 100 including a treatment planning computing system including a mesh cleanup application utilizing a machine learning architecture is shown, according to an embodiment. The system 100 includes user device 121 and treatment planning computing system 110. Devices and components in FIG. 1 can be added, deleted, integrated, separated, and/or rearranged in various embodiments of the disclosed inventions. In some embodiments, operations may be performed on the user device 121. For example, the user device 121 may clean the captured data. However, in other embodiments, operations may be performed on the treatment planning computing system 110. For example, the user device 121 may capture an image, and subsequently transmit the image to the treatment planning computing system 110 for processing. For ease of description, the treatment planning computing system 110 is described as executing the mesh cleanup application. It should be appreciated that the user device 121 and/or some combination of the user device 121 and the treatment planning computing system 110 may execute one or more operations of the mesh cleanup application. Components of the user device 121 and/or treatment planning computing system 110 may be locally installed (on the user device 121 and/or treatment planning computing system 110), and/or may be remotely accessible (e.g., via a browser based interface or a cloud system).

The various systems and devices may be communicatively and operatively coupled through a network 101. Network 101 may permit the direct or indirect exchange of data, values, instructions, messages, and the like (represented by the arrows in FIG. 1). The network 101 may include one or more of the Internet, cellular network, Wi-Fi, Wi-max, a proprietary network, or any other type of wired or wireless network of a combination of wired or wireless networks. The network 101 may facilitate communication between the respective components of the system 100, as described in greater detail below.

The user 120 may be any person using the user device 121. Such a user 120 may be a potential customer, a customer, client, patient, or account holder of an account stored in treatment planning computing system 110 or may be a guest user with no existing account. The user device 121 includes any type of electronic device that a user 120 can access to communicate with the treatment planning computing system 110. For example, the user device 121 may include watches (e.g., a smart watch), and computing devices (e.g., laptops, desktops, personal digital assistants (PDAs), mobile devices (e.g., smart phones)).

The treatment planning computing system 110 may be associated with or operated by a dental institution (e.g., a dentist or an orthodontist, a clinic, a dental hardware manufacturer). The treatment planning computing system 110 may maintain accounts held by the user 120, such as personal information accounts (patient history, patient issues, patient preferences, patient characteristics). The treatment planning computing system 110 may include server computing systems, for example, comprising one or more networked computer servers having a processor and non-transitory machine readable media. In some embodiments, the treatment planning computing system 110 may include a plurality of servers, which may be located at a common location (e.g., a server bank) or may be distributed across a plurality of locations.

As shown, both the user device 121 and the treatment planning computing system 110 may include a network interface (e.g., network interface 124A at the user device 121 and network interface 124B at the treatment planning computing system 110, hereinafter referred to as “network interface 124”), a processing circuit (e.g., processing circuit 122A at the user device 121 and processing circuit 122B at the treatment planning computing system 110, hereinafter referred to as “processing circuit 122”), an input/output circuit (e.g., input/output circuit 128A at the user device 121 and input/output circuit 128B at the treatment planning computing system 110, hereinafter referred to as “input/output circuit 128”), an application programming interface (API) gateway (e.g., API gateway 123A at the user device 121 and API gateway 123B at the treatment planning computing system 110, hereinafter referred to as “API gateway 123”), and an authentication circuit (e.g., authentication circuit 117A at the user device 121 and authentication circuit 117B at the treatment planning computing system 110, hereinafter referred to as “authentication circuit 117”). The processing circuit 122 may include a memory (e.g., memory 119A at the user device 121 and memory 119B at the treatment planning computing system 110, hereinafter referred to as “memory 119”) and a processor (e.g., processor 129A at the user device 121 and processor 129B at a server, hereinafter referred to as “processor 129”).

The network interface circuit 124 may be adapted for and configured to establish a communication session via the network 101 between the user device 121 and the treatment planning computing system 110. The network interface circuit 124 includes programming and/or hardware-based components that connect the user device 121 and/or treatment planning computing system 110 to the network 101. For example, the network interface circuit 124 may include any combination of a wireless network transceiver (e.g., a cellular modem, a Bluetooth transceiver, a Wi-Fi transceiver) and/or a wired network transceiver (e.g., an Ethernet transceiver). In some arrangements, the network interface circuit 124 includes the hardware and machine-readable media structured to support communication over multiple channels of data communication (e.g., wireless, Bluetooth, near-field communication, etc.).

Further, in some arrangements, the network interface circuit 124 includes cryptography module(s) to establish a secure communication session (e.g., using the IPSec protocol or similar) in which data communicated over the session is encrypted and securely transmitted. In this regard, personal data (or other types of data) may be encrypted and transmitted to prevent or substantially prevent the threat of hacking or unwanted sharing of information.

To support the features of the user device 121 and/or treatment planning computing system 110, the network interface circuit 124 provides a relatively high-speed link to the network 101, which may be any combination of a local area network (LAN), the Internet, or any other suitable communications network, directly or through another interface.

The input/output circuit 128A at the user device 121 may be configured to receive communication from a user 120 and provide outputs to the user 120. Similarly, the input/output circuit 128B at the treatment planning computing system 110 may be configured to receive communication from an administrator (or other user such as a medical professional, such as a dentist, orthodontist, dental technician, or administrator) and provide output to the user. For example, the input/output circuit 128 may capture user interaction with a light sensor, user interaction with an accelerometer, and/or user interaction with a camera. For instance, a user 120 using the user device 121 may capture an image of the user 120 using a camera. Moreover, light sensors on the user device 121 can collect data used to determine whether the user device 121 is facing light, and a user 120 may interact with an accelerometer such that the measurement data produced by the accelerometer is used to determine an orientation of the user 120 and the user device 121. Additionally or alternatively, an administrator using the treatment planning computing system 110 may interact with the treatment planning computing system 110 using voice, a keyboard/mouse (or other hardware), and/or a touch screen.

In some embodiments, the camera may be a “front-facing camera.” For example, a front-facing camera may be a camera that is on a front of the user device 121 such that the user 120 may view the display of the user device 121 while facing the camera. In other embodiments, the camera may be a “backward-facing camera.” A backward-facing camera may be a camera that is on a back of the user device 121 such that the user 120 may not view the display of the user device 121 and face the backward-facing camera at the same time. In some embodiments, the backward-facing camera is the same type of camera as the front-facing camera. In other embodiments, the backward-facing camera may be configured to take higher quality images and/or record higher quality video streams than the front-facing camera. The image of the user captured by the camera (either the front-facing camera or the backward facing camera) may be ingested by the user device 121 using the input/output circuit 128.

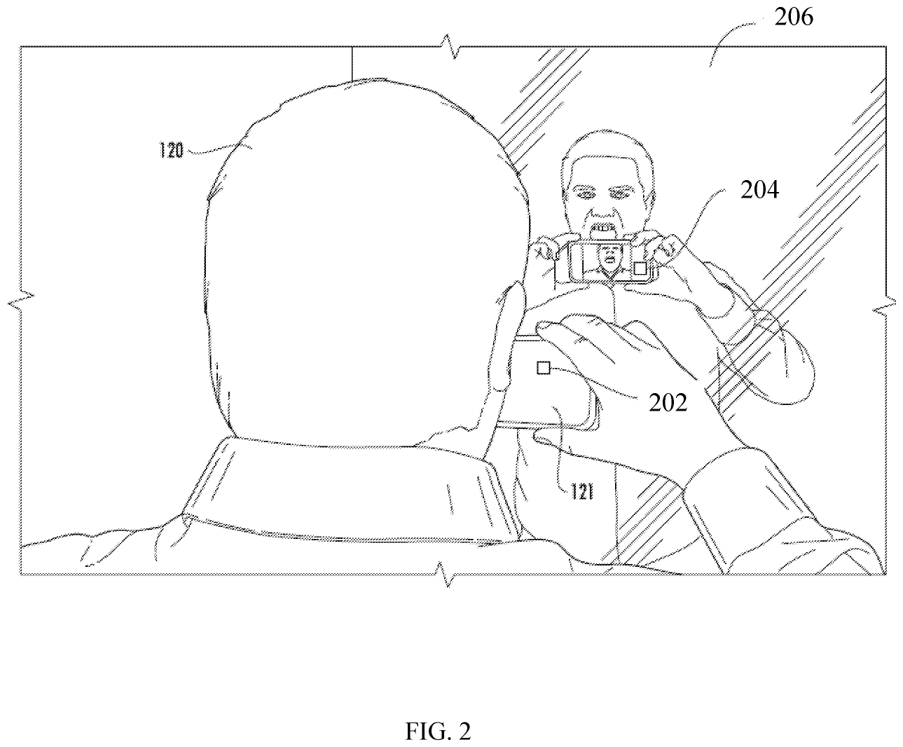

Referring to FIG. 2, depicted is an example of a user using front-facing cameras and rear-facing cameras on a user device. The camera used to capture the image of the user 120 may be a “front-facing” camera 204. For example, a front-facing camera 204 may be a camera that is on a front of the user device 121 (e.g., the same side as a display of a smartphone) such that the user 120 may view the display of the user device 121 while facing the camera. In other embodiments, the camera used to capture the image of the user 120 may be a “rear-facing” camera 202. A rear-facing camera 202 may be a camera that is on a back of the user device 121 (e.g., the side opposite the display of a smartphone) such that the user 120 may not be able to view the display of the user device 121 and face the rear-facing camera 202 at the same time. In some embodiments, the rear-facing camera 202 is the same type of camera as the front-facing camera 204. In other embodiments, the rear-facing camera 202 may be configured to take higher quality images and/or record higher quality video streams than the front-facing camera 204. The image of the user captured by the camera (either the front-facing camera 204 or the rear-facing camera 202) may be ingested by the user device 121 using the input/output circuit 128.

The rear-facing camera 202 and the front-facing camera 204 may respectively comprise a single camera or multiple cameras (e.g., one camera, two cameras, three cameras, four cameras, etc.). The cameras may be the same type of camera or the cameras may be different types of cameras. For example, any camera may be or include one or more of the following features: a wide lens, an ultra-wide lens, a telephoto lens, at least 2× optical zoom, at least 5× digital zoom, and lens correction. Video capabilities of the rear-facing camera 202 and the front-facing camera 204 may include ProRes video recording at least 4K at 30 fps (at least 1080p at 30 fps), HDR video recording with Dolby Vision at least 4K at 60 fps, 4K video recording at 24 fps or 25 fps or 30 fps or 60 fps, at least 1080p HD video recording at 25 fps or 30 fps or 60 fps, at least 1080p HD video recording at 25 fps or 30 fps or 60 fps, sensor-shift optical image stabilization, at least 2× optical zoom, or at least 3× digital zoom. In some embodiments, the rear-facing camera 202 and the front-facing camera 204 may include one or more sensors, such as a LiDAR sensor or other depth sensing technologies including RGBD images. In some embodiments, the rear-facing camera 202 and the front-facing camera 204 can include a light and can be controlled by the user device 121 to turn on when an image is acquired. For example, the input/output circuit 128 may “flash” the light or turn the light on for a continuous duration (e.g., keep the rear-facing light on while a video is acquired using the rear-facing camera). The input/output circuit 128 may also be configured to instruct a user to manually turn on the flashlight of the user device 121.

In some embodiments, the rear-facing camera 202 includes more cameras than the front-facing camera 204. For example, in one embodiment, the front-facing camera 204 comprises a single camera and the rear-facing camera 202 comprises three cameras. In another example, the front-facing camera 204 is a 10-megapixel camera and one of the rear-facing cameras 202 is a 12-megapixel camera. In another example, the front-facing camera 204 is at least a 12-megapixel standard lens camera and the rear-facing camera 202 comprises at least a 12-megapixel wide camera, a 12-megapixel ultra-wide camera, and a 12-megapixel telephoto camera. As such, the rear-facing camera 202 may be capable of obtaining higher quality images than the front-facing camera 204 (e.g., higher resolution, more pixels per inch, higher dynamic range, etc.).

In some embodiments, when the rear-facing camera 202 comprises more than one camera, the input/output circuit 128 is configured to select which camera to use for capturing images or video. The input/output circuit 128 may be configured to select and control the camera capable of obtaining the highest resolution images or video. For example, the input/output circuit 128 may select and control the camera capable of taking the highest quality images based on a characteristic of the camera (e.g., highest resolution, greatest dynamic range, a feature such as lens correction, etc.).

The user 120 may receive feedback when capturing images to improve the quality of a captured image. Generating feedback using user device 121 and/or treatment planning computing system 110 is described in more detail in U.S. patent application Ser. No. 17/401,053, titled “Machine Learning Architecture for Imaging Protocol Detector,” filed Aug. 12, 2021, and U.S. patent application Ser. No. 17/581,811, titled “Machine Learning Architecture for Imaging Protocol Detector,” filed Jan. 21, 2022, the contents of each of which are hereby incorporated by reference in their entirety.

The API gateway 123 may be configured to facilitate the transmission, receipt, authentication, data retrieval, and/or exchange of data between the user device 121, and/or treatment planning computing system 110.

Generally, an API is a software-to-software interface that allows a first computing system of a first entity (e.g., the user device 121) to utilize a defined set of resources of a second (external) computing system of a second entity (e.g., the treatment planning computing system 110, or a third party) to, for example, access certain data and/or perform various functions. In such an arrangement, the information and functionality available to the first computing system is defined, limited, or otherwise restricted by the second computing system. To utilize an API of the second computing system, the first computing system may execute one or more APIs or API protocols to make an API “call” to (e.g., generate an API request that is transmitted to) the second computing system. The API call may be accompanied by a security or access token or other data to authenticate the first computing system and/or the particular user 120. The API call may also be accompanied by certain data/inputs to facilitate the utilization or implementation of the resources of the second computing system, such as data identifying users 120 (e.g., name, identification number, biometric data), accounts, dates, functionalities, tasks, etc.

The API gateway 123 in the user device 121/treatment planning computing system 110 provides various functionality to other systems and devices through APIs by accepting API calls via the API gateway 123. The API calls may be generated via an API engine of a system or device to, for example, make a request from another system or device. For example, the mesh cleanup application 125 at the treatment planning computing system 110 and/or a downstream application operating on the treatment planning computing system 110 may use the API gateway 123B to communicate with user device 121 and/or other third party devices. For example, the mesh cleanup application 125 may query one or more third party devices for training data (e.g., historic images of user mouths, manually sculpted 3D models).

The authentication circuit 117 of the treatment planning computing system 110 may be or include any device(s), component(s), circuit(s), or other combination of hardware components designed or implemented configured to authenticate a user by authenticating information received by the user device 121 and/or treatment planning computing system 110. In some embodiments, the authentication circuit 117 may prompt the user (e.g., user of the user device 121 and/or user of the treatment planning computing system 110) to enter user credentials (e.g., username, password, security questions, and biometric information such as fingerprints or facial recognition). The authentication circuit 117 may look up and match the information entered by the user to stored/retrieved user information in memory 119. For example, memory 119 may contain a lookup table matching user authentication information (e.g., name, home address, IP address, MAC address, phone number, biometric data, passwords, usernames) to a particular user account.

The authentication circuit 117 may authenticate a user 120 as having a user account associated with the treatment planning computing system 110. The user account (or user profile) may be an account associated with a particular user and identify user information and/or medical information. For example, the user account may identify the user's name, email address, home address, biometric data, user name, passwords, feedback preferences, gender, age, purchase history (e.g., purchased aligners), treatment history (e.g., saved treatment plans and any associated information such as initial teeth positions and/or final teeth positions), dental conditions and/or characteristics. Characteristics may include characteristics of the user's detention. For example, a characteristic of the user's detention may be that the user is missing one or more teeth (or particular regions of teeth), the presence of one or more cavities, and the like.

The authentication circuit 117 may also authenticate an administrator (not shown) as having various privileges. For example, an administrator may have an administrative account with specified privileges defined by data fields in user records stored in the memory 119B of the treatment planning computing system 110. The authentication circuit 117 may authenticate the user and identify the user's role/privileges by executing an access directory protocol (e.g., LDAP). The treatment planning computing system 110 may display various parameters to tune the mesh cleanup application 125 according to the user's role. For example, authenticated users with specified user rights may manually sculpt meshes for use in training data to recalibrate the machine learning models executed by the mesh cleanup application.

The processing circuit 122 may include at least memory 119 and a processor 129. The memory 119 includes one or more memory devices (e.g., RAM, NVRAM, ROM, Flash Memory, hard disk storage) that store data and/or computer code for facilitating the various processes described herein. The memory 119 may be or include tangible, non-transient volatile memory and/or non-volatile memory. The memory 119 stores at least portions of instructions and data for execution by the processor 129 to control the processing circuit 122. For example, memory 119 may serve as a repository for user accounts (e.g., storing user name, email address, physical address, phone number, medical history), training data, thresholds, weights, and the like for the machine learning models. In other arrangements, these and other functions of the memory 119 are stored in a remote database.

The processor 129 may be implemented as a general-purpose processor, an application specific integrated circuit (ASIC), one or more field programmable gate arrays (FPGAs), a digital signal processor (DSP), a group of processing components, or other suitable electronic processing components.

The user device 121 and/or treatment planning computing system 110 are configured to run a variety of application programs and store associated data in a database of the memory 119. One such application executed by the user device 121 and/or treatment planning computing system 110 using the processing circuit 122 may be the mesh cleanup application 125. As mentioned earlier, for ease of description, the treatment planning computing system 110 is described as executing the mesh cleanup application 125. It should be appreciated that some portion of the treatment planning computing system 110 and/or the user device 121 may execute the mesh cleanup application 125. Additionally, or alternatively, the mesh cleanup application 125 may be executed completely by the user device 121 (or treatment planning computing system 110), and in some implementations may be run subsequently at the treatment planning computing system 110 (or user device 121).

The mesh cleanup application 125 is configured to automatically remove artifacts and holes, smooth meshes, and generally improve the details and accuracy of dental features of a 3D model. The output of the mesh cleanup application 125 is a 3D surface mesh that closely (e.g., accurately) represents the user's teeth/gums such that the cleaned mesh representation of the user's mouth accurately represents the scanned/captured actual representation of the user's mouth.

The mesh cleanup application 125 is a downloaded and installed application that includes program logic stored in a system memory (or other storage location) of the user device 121 that includes a reception circuit 135, a mesh processing circuit 133, and a mesh output circuit 137 to receive a captured representation of a user's mouth, generate a mesh representation and clean the surfaces/polygons of the mesh representation, and output the cleaned (or enhanced) representation of the user's mouth. In this embodiment, the reception circuit 135, mesh processing circuit 133, and mesh output circuit 137 are embodied as program logic (e.g., computer code, modules, etc.). In some embodiments, during download and installation, the mesh cleanup application 125 is stored by the memory 119B of the treatment planning computing system 110 and selectively executable by the processor 129B. The program logic may configure the processor 129 (e.g., processor 129A of the user device 121 and processor 129B of the treatment planning computing system 110) to perform at least some of the functions discussed herein. In some embodiments the mesh cleanup application 125 is a stand-alone application that may be downloaded and installed on the user device 121 and/or treatment planning computing system 110. In other embodiments, the mesh cleanup application 125 may be a part of another application.

The depicted downloaded and installed configuration of the mesh cleanup application 125 is not meant to be limiting. According to various embodiments, parts (e.g., modules, etc.) of the mesh cleanup application 125 may be locally installed on the user device 121/treatment planning computing system 110 and/or may be remotely accessible (e.g., via a browser-based interface) from the user device 121/treatment planning computing system 110 (or other cloud system in association with the treatment planning computing system 110). In this regard and in another embodiment, the mesh cleanup application 125 is a web-based application that may be accessed using a browser (e.g., an Internet browser provided on the user device). In still another embodiment, the mesh cleanup application 125 is hard-coded into memory such as memory 119 of the user device 121/treatment planning computing system 110 (i.e., not downloaded for installation). In an alternate embodiment, the mesh cleanup application 125 may be embodied as a “circuit” of the treatment planning computing system 110 as a circuit is defined herein.

The mesh cleanup application 125 includes a reception circuit 135. The reception circuit 135 may be or include any device(s), component(s), circuit(s), or other combination of hardware components designed or implemented to determine or otherwise modify a captured digital representation of the user's mouth. The data received by the mesh cleanup application 125 may vary (e.g., 3D scans of the user's mouth, 2D images, etc.). The reception circuit 135 is configured to standardize/normalize the received data for mesh representation enhancement.

The mesh cleanup application 125 may receive 2D images of the user's mouth. For example, the user 120 may start recording a video of the user's mouth. The video of the user's mouth may be considered a continuous stream of data. The reception circuit 135 extracts images from the continuous stream of data by parsing the stream into frames. The frames may be portions or segments of the video data across the time series. For example, at time=0, the reception circuit 135 may capture a static snapshot of the video data (e.g., a frame), at time=2, the reception circuit 135 may capture another static snapshot of the video data. The time between frames may be pre-established or dynamically determined. The time between frames may be static (e.g., frames are captured every 2 seconds) or variable (e.g., a frame is captured 1 second after the previous frame, a next frame is captured 3 seconds after the previous frame, and the like). In some cases, the 2D images of the user's mouth may contain depth data. For example, the images may be RGB-D images if the camera capturing images of the user's mouth is configured to capture RGB-D data.

In other cases, the 2D images of the user's mouth may not contain depth data. In these cases, the reception circuit 135 may employ photogrammetry to extract 3D measurements from the captured 2D image and generate a 3D representation of the user's mouth. For instance, the roll, pitch, yaw and distance of the user's 120 head may be determined using photogrammetry or one or more other algorithms. The reception circuit 135 may perform photogrammetry by comparing known measurements of facial features with measurements of facial features in the image. The facial features measured in the image (e.g., including the lengths/sizes of the various facial features in the image) may include tooth measurements, lip size measurements, eye size measurements, chin size measurements, and the like. The known facial features may be average facial features (including teeth, chin, lips, eyes, nose) stored in one or more databases (e.g., treatment planning computing system 110 memory 119B) and/or from local memory 119A. The known facial features may also be particular measurements of a user (e.g., measured when the user 120 was at a medical professional's office) from local memory 119A and/or a database (e.g., treatment planning computing system 110 memory 119B). The reception circuit 135 compares the known measurements of facial features with dimensions/measurements of the facial features in the image to determine depth information including position, orientation, size, and/or angle of the facial feature in the image.

The reception circuit 135 may also perform triangulation to generate a 3D representation of the user's mouth using images from various perspectives. As an example, the reception circuit 135 may associate a two-dimensional pixel in the received images with a ray in three-dimensional space. Given multiple perspectives of the image (e.g., at least two images capture the position of the user from at least two different perspectives), the reception circuit 135 may determine a three-dimensional point from the intersection of at least two rays from pixels of the received images.

Generating 3D models (including point clouds) from 2D images is described in more detail in U.S. patent application Ser. No. 16/696,468, now U.S. Pat. No. 10,916,053, titled “SYSTEMS AND METHODS FOR CONSTRUCTING A THREE-DIMENSIONAL MODEL FROM TWO-DIMENSIONAL IMAGES” filed on Nov. 26, 2019, and U.S. patent application Ser. No. 17/247,055 titled “SYSTEMS AND METHOD FOR CONSTRUCTING A THREE-DIMENSIONAL MODEL FROM TWO DIMENSIONAL IMAGES” filed on Nov. 25, 2020, where the contents of these applications are incorporated herein by reference in their entirety.

The mesh cleanup application 125 may also receive a 3D representation of the user's mouth. For example, 3D scanning equipment, such as intraoral scanning devices, may capture a 3D representation of the user's mouth and generate a mesh representation from the 3D representation. In some embodiments, the reception circuit 135 is configured to generate a mesh representation from a 3D representation of the user's mouth by partitioning the 3D representation (e.g., the continuous geometric space) into discrete cells. The reception circuit 135 is configured to employ one or more mesh generation models to create the 3D mesh representation (e.g., the polygons with surfaces having an orientation in a three dimensional space).

The reception circuit 135 may perform one or more preprocessing operations including normalizing the 2D/3D representation of the user's mouth, scaling the 2D/3D representation of the user's mouth, rotating the 2D/3D representation of the user's mouth, and the like. In some implementations, the reception circuit 135 may extract features from the 2D/3D representation of the user's mouth. For example, the reception circuit 135 may perform feature extraction by applying convolution to an image and generating a feature map of extracted features. Convolving the image with a filter (e.g., kernel) has the effect of reducing the dimensionality of the image.

Additionally, or alternatively, the reception circuit 135 may down sample the feature map (or the digital representation of the user's mouth) by performing pooling operations. Pooling is employed to detect maximum features, minimum features, and the like from a pooling window of a predetermined length/duration. In an example, a maximum pooling window is configured to extract the maximum features of the feature map (e.g., the prominent features having higher relative values in the pooling window). In some configurations, the reception circuit 135 may include a flattening operation, in which the reception circuit 135 arranges a feature map (represented as an array) into a one-dimensional vector.

The reception circuit 135 may also perform pose estimation on the representation of the user's mouth. For example, the reception circuit may perform bottom-up pose estimation approaches and/or top-down pose-estimation approaches. The reception circuit 135 may implement an autoencoder trained to estimate landmarks on an image. The reception circuit 135 performs pose estimation to identify localized sets of landmarks in an image or video frame. The sets of landmarks may be considered a landmark model. For example, there may be a landmark model of a face, a landmark model of a mouth, etc.

The landmarks may indicate coordinates, angles, and features relevant to head angles, mouth angles, jaw angles, and/or visibility of the teeth in the representation of the user's mouth. The pose estimation may be performed at various levels of granularity. For example, in some embodiments, the reception circuit 135 may landmark a face. In some embodiments, the reception circuit 135 may landmark a chin on the face, a nose on the face, etc.

The mesh cleanup application 125 also includes a mesh processing circuit 133. The mesh processing circuit 133 may be or include any device(s), component(s), circuit(s), or other combination of hardware components designed or implemented to clean and sculpt a mesh representation. If the mesh processing circuit 133 receives a 3D representation of the user's mouth from the reception circuit 135 (e.g., the reception circuit 135 converts a 2D image into a 3D model, the reception circuit 135 receives a 3D scan or a 3D mesh, etc.), the mesh processing circuit 133 is configured to map the received 3D representation to 2D image space. In some implementations, the mesh processing circuit 133 uses representation into 2D image space into 2D image spacemesh processing circuit 133 to differentiate textures of the user's mouth (e.g., differentiate teeth from gums, differentiate teeth from other teeth), which is important when the mesh processing circuit 133 reconstructs a 3D surface from 2D space. As part of the UV mapping, the mesh processing circuit 133 encodes local surface characteristics, including, but not limited to, vertex normal, face normal, curvature, multi-dimensional representations or high dimensional representations, embeddings of the plurality of surfaces using the channels of the captured image (i.e., image channels of the 2D representation). For example, encoding the local 3D surface characteristics could include encoding each face normal of the 3D representation as RGB values (i.e., transfer the labels on the mesh to their corresponding pixels, to create a 3-channel image). In some implementations, additional descriptors other than face normals can be used to represent a mesh. For example, descriptors for 5-channel, 30-channel, or 50-channel images can be used.

A 3D model in a mesh representation may be considered a collection of polygons (or meshes), where each surface of the polygon includes vertices, edges and faces that define the shape of the polygon. The mesh processing circuit 133 may determine one or more surface characteristics for each surface, where the face (or vertex) normal can be defined as a unit vector extending in the x, y, and z-directions (e.g., sometimes normal to the surface face). Encoding the surface characteristics of each face allows the mesh processing circuit 133 to track the orientation of each face in space (e.g., the x, y, z-direction). For example, the mesh processing circuit 133 may create a vector encoded with RGB values for each edge (or face or vertex).

In some implementations, a 3D model can be represented as a point cloud, an assembly of data points in three-dimensional space, each defined by X, Y, and Z coordinates. Similar to polygons, the mesh processing circuit 133 can calculate and encode the 3D surface characteristics of each point, allowing tracking of the orientation of each point in space, for instance by creating a vector encoded with surface characteristics for each point in the cloud.

In some implementations, a 3D model can be represented as voxel-based structures, where the model is described as a three-dimensional grid of volume pixels or voxels. Similar to the polygons and point clouds, the mesh processing circuit 133 could encode and track the orientation (e.g., surface characteristics) of each voxel in space, for instance, by attributing an encoded vector with surface characteristics to each voxel.

In some implementations, a 3D model can be represented as parametric surfaces such as NURBS (Non-Uniform Rational B-splines). In this implementation, the 3D model is described by a mathematical function, and the mesh processing circuit 133 could compute and encode the orientation (e.g., surface characteristics) at any given point on the surface, for example, by attributing an encoded vector with 3D surface characteristics to some sampling of points from the surface describing function for each point on the surface. To sample points from the parametric surface, the mesh processing circuit 133 may evaluate the mathematical function defining the NURBS at a specific set of parameters. For example, variables u and v might each vary from 0 to 1, and the mesh processing circuit 133 could evaluate the NURBS function at regular intervals (e.g., 0, 0.1, 0.2, . . . , 1) for both u and v to generate a grid of points on the surface. This sampled grid of points from the parametric surface can then be used to construct a polygonal mesh representation of the surface. In some embodiments, adjacent points on the grid can be connected by edges to form a collection of polygons (typically quadrilaterals or triangles), which approximate the geometry of the original parametric surface. Additionally, the mesh processing circuit 133 may also calculate and store additional information at each sampled point, such as the normal vector to the surface, which describes the orientation of the surface at that point.

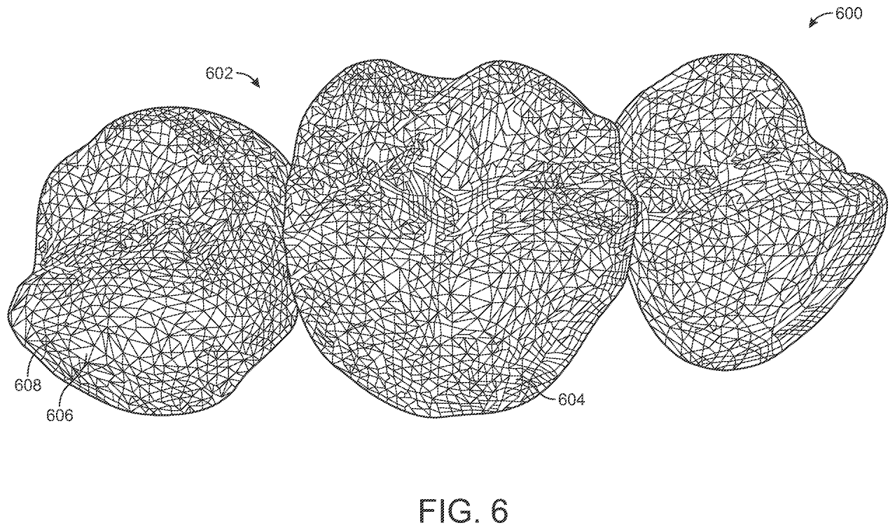

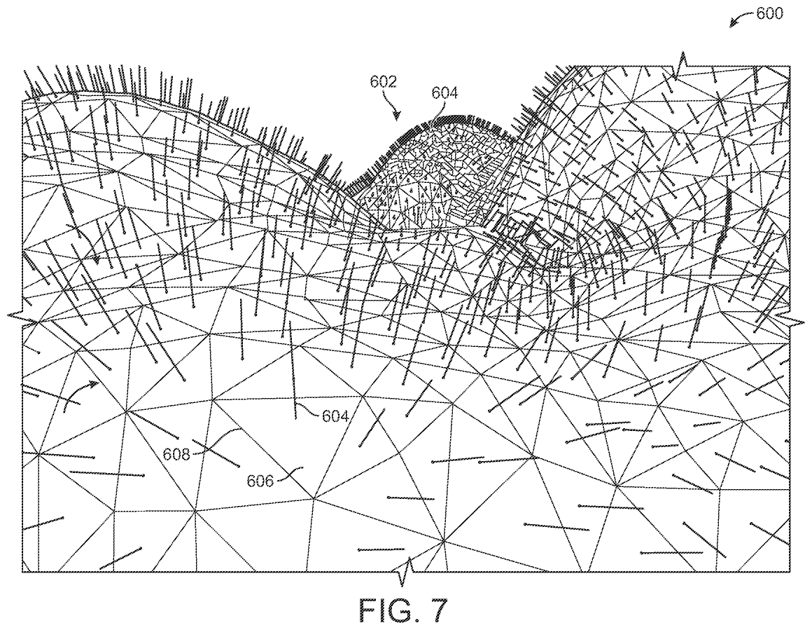

Referring to FIG. 6, depicted is a mesh representation of a user's teeth. The mesh representation 600 is a digital model 602 of a user's teeth 604 (e.g., the right or left rear three teeth). As shown in FIG. 6, the digital model 602 could be formed from a large number of polygons 606, connected at edges 608. More specifically, referring to FIG. 7, the mesh representation 600 displaying the digital model 602 is shown from another view. As illustrated in FIG. 7, the model is formed from polygons 606 connected at edges 608. Additionally, a face normal 614 has been computed for each polygon 606, and these face normals 614 are also displayed in FIG. 7.

While FIGS. 6-7 depict the digital model 602 as being formed from a large number of interconnected polygons 606, it should be understood that this is not the only way to represent the surfaces of the digital model 602, such as a mesh. In some implementations, the surfaces could be parameterized with a function, such as, but not limited to, a Non-Uniform Rational B-splines (NURBS), ray tracing, Bezier surfaces, B-spline surfaces, subdivision surfaces, or implicit surfaces defined by mathematical equations. For example, ray tracing could be employed to simulate the trajectory of light rays from each pixel into the scene to determine their points of intersection with surfaces. In another example, using NURBS, surfaces can be represented as a set of smoothly connected curves to model organic shapes, such as teeth or other body parts (e.g., of a human or animal (e.g., dog, cat, horse)).

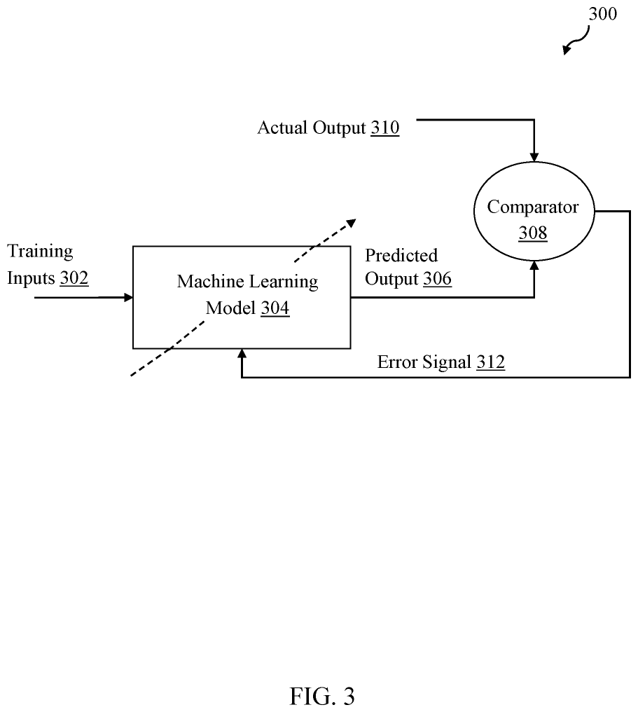

The mesh processing circuit 133 is also configured to apply a machine learning model to the representation of the user's mouth to clean the representation. Training a supervised machine learning model to clean the representation of the user's mouth and accurately sculpt the user's mouth is described in more detail with reference to FIG. 3.

Referring to FIG. 3, a block diagram of an example system 300 using supervised learning to clean and sculpt meshes is shown according to an example embodiment. Supervised learning is a method of training a machine learning model given input-output pairs. An input-output pair is an input with an associated known output (e.g., an expected output, a labeled output). The machine learning model 304 may be trained on known input-output pairs (e.g., representations of a user's mouth and manually sculpted/cleaned representations of the user's mouth) such that the machine learning model 304 learns how to predict known outputs given known inputs. Once the machine learning model 304 has learned how to predict known input-output pairs, the machine learning model 304 can operate on unknown inputs to predict an output.

To train the machine learning model 304 using supervised learning, training inputs 302 and actual outputs 310 may be provided to the machine learning model 304. The machine learning model 304 may any machine learning model such as a convolutional neural network, a UNet, Generative Adversarial Networks (GANs), and the like. Training inputs 302 may include a representation of a user's mouth (e.g., 3D surface meshes, 3D surface meshes mapped into 2D space using UV mapping, 2D images of a user's mouth, 2D images of a user's mouth mapped to a 3D representation, a 2D image of the user's mouth mapped to a 3D representation and subsequently mapped into a 2D space using UV mapping). The training inputs 302 may be selected from a database of historic representations of mouths captured from patients, professional medical imaging equipment, and the like. Actual outputs 310 may be manually sculpted and cleaned mesh representations of the user's mouth that correspond to the training inputs 302. One or more highly skilled and trained clinicians, physicians, orthodontists, dentists and the like may manually manipulate mesh representations corresponding to the training inputs 302 using software. The manipulated mesh representations accurately represent the user's mouth in 3D mesh space such that that digital representation of the user's teeth is representative of the shape, size, orientation, position, etc., of the actual user's teeth. In some embodiments, the mesh processing circuit 133 maps the actual outputs 310 into 2D space using UV mapping such that the dimension of the training inputs 302 is the same as the dimension of the actual outputs 310 (e.g., 2D space vs 3D space). For example, if the training inputs 302 is in the form of a 3D mesh, the mesh processing circuit 133 may not map the corresponding sculpted 3D mesh representation (e.g., the actual output 310) into 2D space using UV mapping. In this manner, the machine learning model may be trained using 3D input-output pairs.

During training, the mesh processing circuit 133 may select representations of the user's mouth (e.g., training inputs 302) and corresponding representations of the user's mouth that have been manually cleaned and sculpted (e.g., actual outputs 310) from one or more databases. The mesh processing circuit 133 may select the training inputs 302 and corresponding actual outputs 310 randomly, in a sequence, as part of a partitioned dataset, or according to one or more bias factors. A bias factor is a factor that biases the mesh processing circuit 133 to select (or avoid) particular input-output pairs (e.g., training inputs 302 and corresponding actual outputs 310).

For example, the mesh processing circuit 133 may identify, and select for training (e.g., oversample) input-output pairs that are associated with one or more flags. The mesh processing circuit 133 may flag input-output pairs if the output (e.g., the manually sculpted/cleaned mesh representation) and/or the input (e.g., the representation of the user's mouth received from the reception circuit 135) are associated with a threshold customer satisfaction score, a threshold number of mouth conditions, a threshold score of a mouth condition, or some combination.

A customer satisfaction score is determined according to a customer (or user 120) experience. In a non-limiting example, dental aligners may be manufactured for the user using the manually cleaned/sculpted mesh representation (e.g., the actual output 310). In response to wearing/receiving the manufactured dental aligners, the user may review the dental aligners. For example, the user may interact with an application executed by the user device 121 and score the dental aligners (e.g., comfort, aesthetics, fit, etc.). The application on the user device 121 may transmit the score to the treatment planning computing system 110 such that the mesh representation is scored implicitly based on the score of the dental aligner. If the mesh representation receives a customer satisfaction score satisfying a threshold (e.g., a high score, a low score), then the mesh cleanup application 125 may flag the manually sculpted mesh representation (e.g., the actual output 310). If the mesh representation is associated with a flag indicating a high customer satisfaction score, then the mesh processing circuit 133 may use the flagged mesh representation (e.g., the actual output 310, the manually sculpted/cleaned representation) and the corresponding input (e.g., the representation of the user's mouth that was cleaned/manually sculpted) as an input-output pair during training of the machine learning model 304. Alternatively, if the mesh representation is associated with a flag indicating a low satisfaction score, then the mesh processing circuit may not use the flagged mesh representation and the corresponding input as an input-output pair during training. The mesh cleanup application 125 may employ one or more thresholds to differentiate “high” customer satisfaction scores and/or “low” customer satisfaction scores.

By prioritizing/oversampling input-output pairs that are associated with high customer satisfaction scores (e.g., flagged manually sculpted representations associated with a customer satisfaction score above a customer satisfaction score threshold), the machine learning model 304 will learn the features/relationships that result in manually sculpted representation receiving high customer satisfaction scores. Learning the features/relationships that resulted in manually sculpted representations receiving high customer satisfaction scores allows the machine learning model 304 to predict cleaned/enhanced representations (e.g., predicted output 306) based on the learned features/relationship. Accordingly, the predicted cleaned/enhanced representations may result in representations that receive high customer satisfaction scores (e.g., a customer satisfaction score above a threshold) if the predicted cleaned/enhanced representations were evaluations by customers/users.

A representation of the user's mouth may be characterized by one or more mouth conditions. For example, a user may be missing a number of teeth. Additionally or alternatively, the user's gingival margin may be refined. When the reception circuit 135 receives the representations of the user mouth (e.g., as 3D scans, images, videos, etc.), the reception circuit 135 may employ object detection/classification algorithms to identify mouth conditions in the representation of the mouth. For example, one or more object detection algorithms may be trained to identify facial features such as teeth (including missing teeth and chipped teeth), lips, tongue, hardware (e.g., mouth piercings, bejeweled teeth) and the like in 3D scans, 2D images, or other digital representations of the user's mouth. In response to identifying one or more mouth conditions, the reception circuit 135 may flag the representation. Additionally, or alternatively, one or more medical professionals may input a flag associated with the representation. For example, a medical professional in an office may flag that a user is missing one or more teeth when the medical professional takes a dental impression of the user's mouth. Additionally, or alternatively, a medical professional manually reviewing captured images of a user's mouth may flag the representation of the user's mouth. The medical professional may flag the representation of the user's mouth for being in a great condition (e.g., no missing teeth) and/or poor condition (e.g., one or more missing teeth, refined gingival margins, etc.), or some variation.

The mesh processing circuit 133 may be configured to train the machine learning model 304 with input-output pairs associated with flags. For example, the mesh processing circuit 133 may oversample input-output pairs that are associated with a flag indicating a positive customer review. Additionally, or alternatively, the mesh processing circuit 133 may determine to not train the machine learning model 304 with cleaned/sculpted mesh representations that are associated with a flag indicating a negative review. If the machine learning model 304 is not trained with input-output pairs that resulted in negative reviews of the mesh representation, then the machine learning model 304 will not learn the features/relationships characteristic of the input-output pairs that resulted in negative reviews. In this manner, the machine learning model 304 does not learn to sculpt/clean mesh representations that result in a negative review. Similarly, the mesh processing circuit 133 may determine to not train the machine learning model 304 with representations of a user's mouth associated with flags indicating one or more mouth conditions. Accordingly, the input-output pairs used to train the machine learning model may be based on an average mouth (e.g., no missing teeth). In this manner, the machine learning model 304 will be tuned to predict an enhanced mesh representation of an average mouth.

In some embodiments, the mesh processing circuit 133 may train the machine learning model 304 using only (or a majority of) flagged input-output pairs. For example, if a database contains enough input-output pairs that are flagged with user mouths characterized by refined gingival margins, then the mesh processing circuit 133 may train the machine learning model 304 specific to representations of user mouths characterized by refined gingival margins. In this manner, instead of training the machine learning model to be tuned to predict an enhanced mesh representation of an average mouth, the machine learning model is trained to sculpt/clean meshes accurately given refined gingival margins. Accordingly, the predicted cleaned/enhanced mesh representation more accurately reflects the actual user's mouth (e.g., the user's mouth with refined gingival margins).

In some embodiments, the flags may be binary (e.g., indicating that the cleaned/sculpted mesh received a positive user review, indicating that the user representation is missing a single tooth). In some embodiments, the flags may be numerical/categorical (e.g., indicating that the cleaned/sculpted mesh received a 95% positive review, indicating that the user representation is missing a top incisor tooth).

The training inputs 302 and actual outputs 310 may be stored in memory or other data structure accessible by the machine learning model 304. In some embodiments, the mesh processing circuit 133 trains the machine learning model 304 using average training data. That is, the training inputs 302 are digital representations (e.g., images, scans) associated with multiple users. Additionally, or alternatively, the mesh processing circuit 133 trains the machine learning model 304 using particular training data. For example, the mesh processing circuit 133 may train the machine learning model 304 according to a single user, regional/geographic users, particular user genders, user's grouped with similar disabilities, users of certain ages, and the like. Accordingly, the machine learning model 304 may be user-specific.

By training the machine learning model using user-specific data (e.g., using input-output pairs associated with a single user, regional/geographic users, particular user genders, user's grouped with similar disabilities, users of certain ages), then the machine learning model 304 will learn the features/relationships associated with the user-specific data. The machine learning model derives relationships/features associated with the user-specific data, allowing the machine learning model 304 to predict cleaned/enhanced representations (e.g., predicted output 306) based on the learned features/relationship associated with the user-specific data. For example, average mouth data may be different from mouth data associated with users in a specific age group. Training the machine learning model based on user-specific age data allows the machine learning model 304 to learn age-specific features and predict enhanced representations based on the learned features. Accordingly, the predicted representation may more closely represent the actual representation of the user's mouth in that particular age group.

The mesh processing circuit 133 may be configured to train multiple machine learning models 304 such that different machine learning models are executed depending on the representation of the user's mouth received from the reception circuit 135. For example, if the mesh processing circuit 133 receives a 3D model from the reception circuit 135, the mesh processing circuit 133 executes a machine learning model trained to receive 3D models and output 3D models. If the mesh processing circuit 133 receives a 3D model with a flag indicating a missing tooth, the mesh processing circuit 133 executes a machine learning model trained to receive 3D models with a missing tooth and output 3D models that represent a mouth representative of a mouth missing a tooth. If the mesh processing circuit 133 receives a 3D model with a flag indicating a particular user age, the mesh processing circuit 133 executes a machine learning model trained to receive 3D models of that user's age group and outputs 3D models representative of that age group's teeth and gums.

The machine learning model 304 may use the training inputs 302 to predict outputs 306 (e.g., a predicted clean/sculpted representation of the user's mouth in 2D space or 3D space), by applying the current state of the machine learning model 304 to the training inputs 302. The comparator 308 compares the predicted outputs 306 to the actual outputs 310 to determine an amount of error or differences.

During training, the error (represented by error signal 312) determined by the comparator 308 may be used to adjust the weights in the machine learning model 304 such that the machine learning model 304 changes (or learns) over time to automatically clean and sculpt representations of the user's mouth in 2D space/3D space. The machine learning model 304 may be trained using the backpropagation algorithm, for instance. The backpropagation algorithm operates by propagating the error signal 312. The error signal 312 may be calculated each iteration (e.g., each pair of training inputs 302 and associated actual outputs 310), batch, and/or epoch and propagated through all of the algorithmic weights in the machine learning model 304 such that the algorithmic weights adapt based on the amount of error. The error is minimized using a loss function. Non-limiting examples of loss functions may include the square error function, the root mean square error function, and/or the cross entropy. Non-limiting examples of loss functions may include the square error function, the room mean square error function, and/or the cross entropy error function.

The weighting coefficients of the machine learning model may be tuned to reduce the amount of error thereby minimizing the differences between (or otherwise converging) the predicted output 306 and the actual output 310. For instance, because a machine learning model is trained to clean and sculpt the representation of the user's mouth in 2D space, the automatically cleaned and sculpted representation of the user's mouth in 2D space will iteratively converge to the manually cleaned and sculpted representation of the user's mouth in 2D space. The mesh processing circuit 133 may train the machine learning model 304 until the error determined at the comparator 308 is within a certain threshold (or a threshold number of batches, epochs, or iterations have been reached). The trained machine learning model 304 and associated weighting coefficients may subsequently be stored in memory or other data repository (e.g., a database) such that the trained machine learning model 304 may be employed on unknown data (e.g., not training inputs 302). Once trained and validated, the machine learning model 304 may be employed during testing (or inference). During testing, the machine learning model ingests unknown data to automatically clean and sculpt a surface mesh representation of the user's mouth. For example, during testing, the machine learning model 304 may ingest a 3D scan of the user's mouth and automatically generate a 3D surface mesh representation of the user's mouth that simulates a 3D surface mesh representation of the user's mouth manually cleaned/sculpted.

Referring back to FIG. 1, when the mesh processing circuit 133 converts a 3D representation into 2D space, the mesh processing circuit 133 is configured to convert the 2D representation back into a 3D representation. The mesh processing circuit 133 may apply UV mapping techniques to reconstruct the mesh in 3D space. The mesh processing circuit 133 preserves the orientation of the meshes in 3D space by encoding the local 3D surface characteristics of a plurality of surfaces using one or more channels of the 2D representation.

The mesh cleanup application 125 also includes a mesh output circuit 137. The mesh output circuit 137 may be or include any device(s), component(s), circuit(s), or other combination of hardware components designed or implemented to determine or otherwise generate a representation of the user's mouth. For example, the mesh output circuit 137 may convert a 3D point cloud representation of the user's mouth into a 3D mesh representation of the user's mouth. Additionally, or alternatively, the mesh output circuit 137 may convert a 3D mesh representation of the user's mouth into a point cloud representation of the user's mouth. The mesh output circuit 137 transforms the representation of the user's mouth into a representation suitable for one or more downstream applications. The mesh output circuit 137 may also be configured to execute one or more post-processing operations including scaling the 2D/3D representation of the user's mouth, rotating the 2D/3D representation of the user's mouth, and the like.

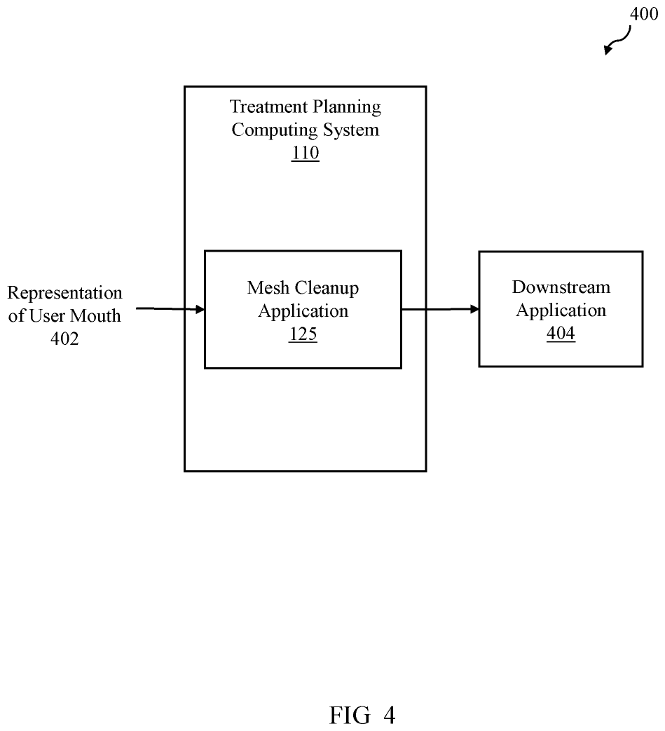

Referring to FIG. 4, a block diagram 400 of an example system including the treatment planning computing system 110 of FIG. 1 configured to clean a digital representation of a user's mouth for a downstream application is shown, accordingly to an illustrative embodiment. The treatment planning computing system 110 receives one or more representations of a user mouth 402. The representations may include 3D models from dental impression kits, 3D models from scanning equipment, 2D images from a camera, and the like. In some implementations, the treatment planning computing system 110 retrieves the representation of the user mouth 402 from a database. The treatment planning computing system 110 executes the mesh cleanup application 125 to clean and sculpt a 3D mesh representation derived from the received representations of the user's mouth 402.

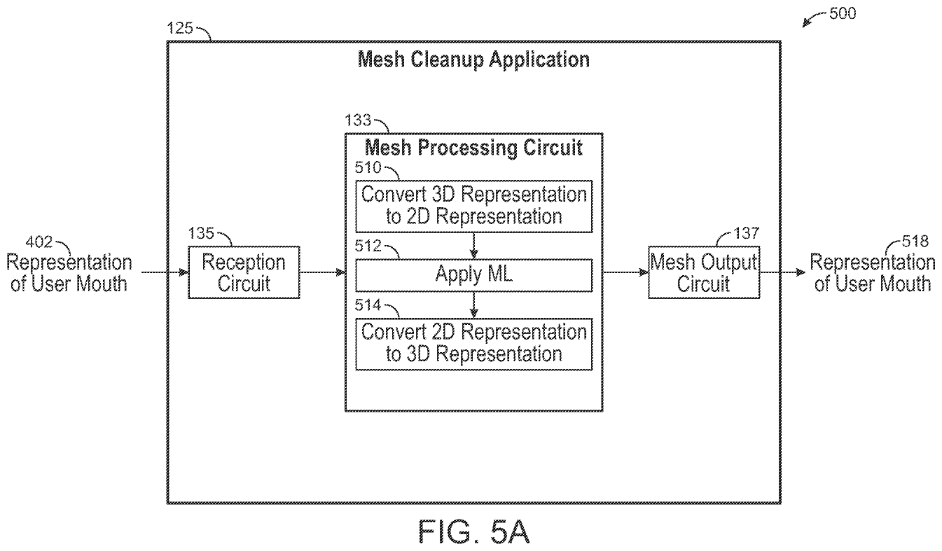

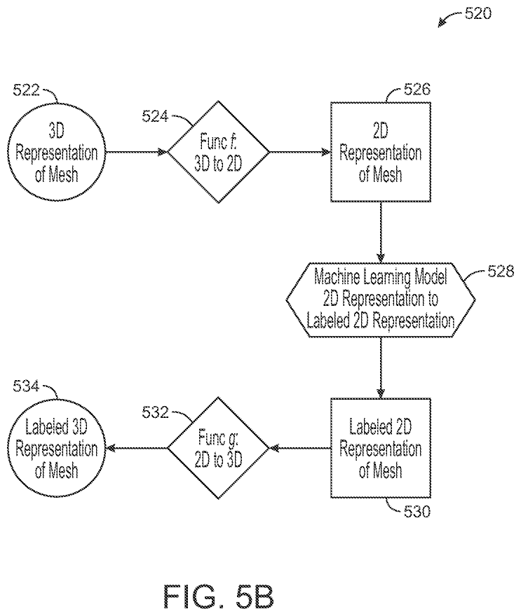

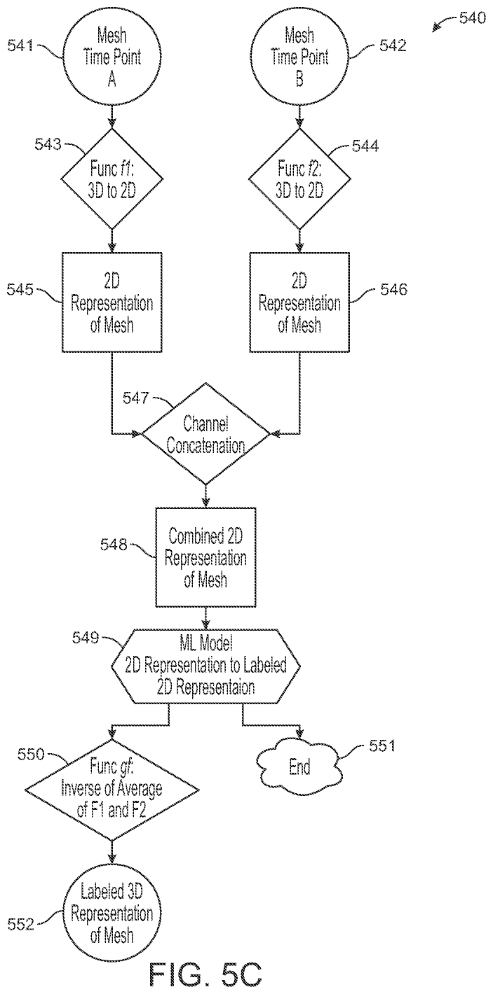

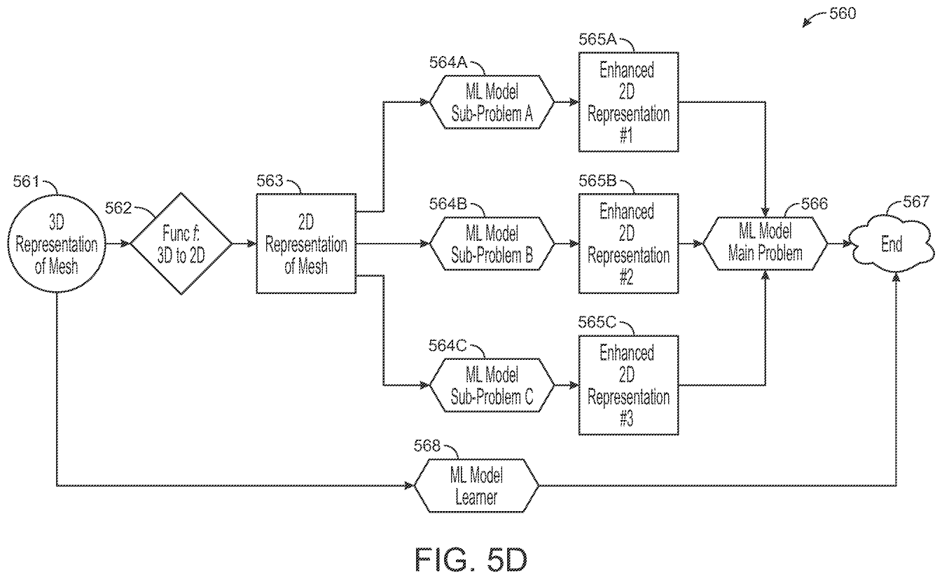

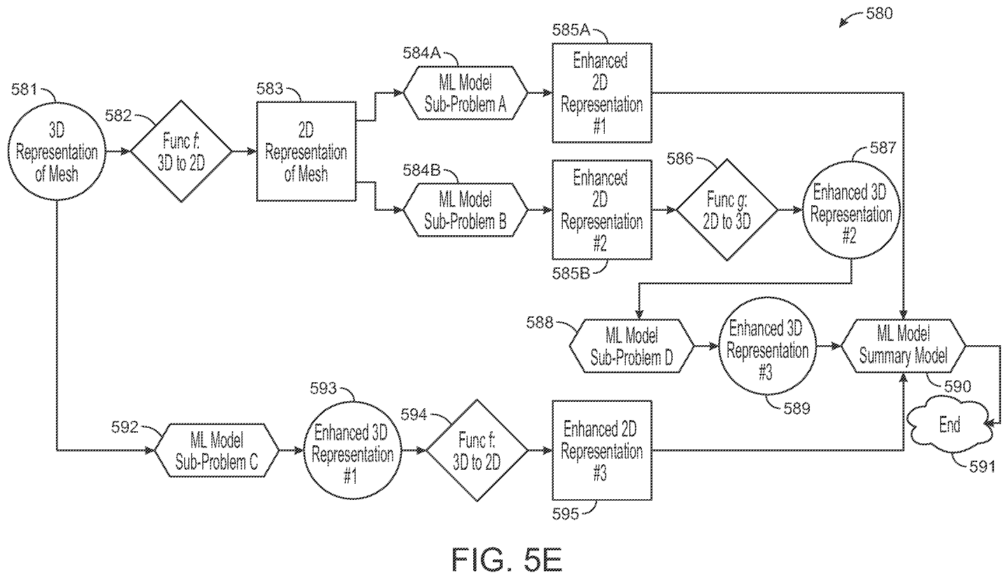

Referring to FIG. 5A, a flow diagram 500 of the mesh cleanup application 125 of FIG. 1 is shown, accordingly to an illustrative embodiment. The operations performed by the mesh cleanup application 125 (including the reception circuit 135, mesh output circuit 137, and the mesh processing circuit 133) vary according to the received representation of the user mouth 402.

As described with reference to FIGS. 4 and 5A, the mesh cleanup application 125 may receive a 2D representation of a user's mouth 402. For example, the representation of the user's mouth may be a stream of data recorded from the user device 121 (or some other device). The reception circuit 135 samples the data stream to capture a single frame of the data stream. In some embodiments, the reception circuit 135 processes each of the frames of the data stream. The frame(s) may be 2D image(s) without depth information. In these embodiments, the reception circuit 135 may extract depth information from an image (e.g., using photogrammetry). The reception circuit 135 may also extract depth information from multiple images using triangulation. Using the depth information and the 2D image, the reception circuit generates a depth image (e.g., a 3D representation of the user's mouth). In some embodiments, the mesh cleanup application 125 receives a representation of the user's mouth 402 with depth information (e.g., 2D images with depth information such as RGB-D images). The mesh cleanup application may also receive a 3D representation of the user's mouth 402. For example, the 3D representation may be a CT image, a model from a dental impression, and the like.

The mesh processing circuit 133 cleans and sculpts the representation of the user's mouth. In step 510, the mesh processing circuit 133 receives the 3D representation of the user's mouth and converts the 3D representation into a 2D representation. As discussed, the mesh processing circuit 133 uses UV mapping to unwrap the 3D representation, assigning pixels to the surface of the 3D model by encoding orientation information (e.g., 3D surface characteristics of a plurality of surfaces) associated with each face. For example, each edge of a face may be encoded with 3-channel values (e.g., RGB). Additionally, or alternatively, faces and/or vertices may be encoded with RGB values. In another example, each surface characteristic may be encoded with 4-channel values (e.g., cyan magenta yellow black (CMYK)). In yet another example, each surface characteristic may be encoded with 5-channel values (e.g., green blue yellow red white (GBYRW)). In yet another example, each surface characteristic may be encoded with 10-channel values (e.g., red, green, blue, alpha, hue, saturation, brightness, X-coordinate, Y-coordinate, and Z-coordinate). It should be understood that any number of channels of the 2D representation (e.g., 3, 4, 5, 20, 30, 50, 100, n, etc.) can be used to describe the local surface characteristics (e.g., of a face, dentition, another body part or portion). Thus, the number of channels in the 2D representation can vary widely, ranging from a 3-channel, 4-channel, 5-channel, 10-channel, n-channel representation, or any other number of channels, as needed to accurately describe the local surface characteristics of a face, dentition, another body part, or portion, depending on the application's requirements and the specific encoding scheme employed (e.g., RGB, CMYK, GBYRW, XYZ coordinates, etc.).

In step 512, the mesh processing circuit 133 applies a trained machine learning model (e.g., machine learning model 304 in FIG. 3) to clean and sculpt the 2D representation of the user's mouth. The trained machine learning model simulates the cleaning and sculpting performed manually by a professional. The result of the machine learning model is an accurate representation of the user's mouth in 2D space.

In step 514, the mesh processing circuit 133 converts the 2D representation of the user's mouth into a 3D representation of the user's mouth. For example, the mesh processing circuit 133 uses UV mapping to convert the 2D representation back to a 3D representation. The mesh processing circuit 133 is able to convert the 2D representation to the 3D representation using the encoded 3D surface characteristics.

In some implementations, the mesh processing circuit 133 does not perform steps 510 and 514 (e.g., converting a 3D representation to a 2D representation and subsequently converting the 2D representation back to the 3D representation). The mesh processing circuit 133 may not perform steps 510 and 514 if the user representation of the mouth is in 3D space and the machine learning model is configured to receive 3D representations. For example, the machine learning model applied in step 512 may be a PointNet or PointNet++. The machine learning model may be trained to clean/enhance representations of the user's mouth in 3D space based on input-output pairs in 3D space. For example, referring to FIG. 3, the training inputs 302 may be 3D scans of a user's mouth and the actual outputs 310 may be corresponding manually sculpted 3D representations of the user's mouth. In these implementations, the reception circuit 135 may transform the representation of the user mouth 402 into a point cloud using a neural network, as described with reference to U.S. Pat. No. 10,916,053, which is incorporated herein by reference in its entirety as noted above. Additionally, or alternatively, the mesh cleanup application 125 receives a point cloud as a representation of the user mouth 402 (e.g., equipment employing LiDAR may produce a point cloud representation of the user mouth 402).

The mesh output circuit 137 is configured to receive the representation of the user mouth from the mesh processing circuit 133. The mesh output circuit 137 transforms the representation of the user mouth into a representation of the user mouth 518 for suitable one or more downstream applications 404.

Referring back to FIG. 4, downstream application 404 receives the representation of the user mouth 518 from the mesh cleanup application. Downstream applications 404 may include applications executed on the user device 121, the treatment planning computing system 110, and/or a third party device. In some embodiments, downstream applications executed on the treatment planning computing system 110 may be applications that may be performed offline or may be associated with high latency (e.g., the user 120 may wait several minutes, hours, days, or weeks before receiving results from the downstream application).

The downstream application 404 may be an application that incorporate control systems (e.g., using a proportional integral derivative (PID)) controllers. A PID controller may be a controller that uses a closed loop feedback mechanism to control variables relating to the image capture process. For example, the PID controller may be used to control an input/output circuit 128 (e.g., a generate instructions to move or autofocus a camera at the user device 121).

The downstream application 404 may also generate a treatment plan (e.g., a series of steps used to correct or otherwise modify the positions of the user's teeth from an initial position to a final position or other intermediary positions) using captured representations of the user's mouth. For example, the downstream application 404 may determine a parametric model from captured images. The downstream application 404 may manipulate one or more parametric models of individual teeth. The manipulation may be performed manually (e.g., based on a user input received via the downstream application 404), automatically (e.g., by snapping/moving the teeth parametric model(s) to a default dental arch), or some combination. The manipulation may include lateral/longitudinal movements, rotation movements, translational movements, etc. The result of the manipulated parametric model(s) may result in a final (or target) position of individual teeth of the user following treatment via dental aligners.

The downstream application 404 may be configured to generate a treatment plan based on the initial position (e.g., the initial position of the user's teeth indicated in the model corresponding to the portions of teeth from the captured high quality image(s)) and the final position (e.g., the final position of the user's teeth following manipulation of the parametric model(s) and any optional adjustments). The downstream application 404 may also generate one or more intermediate stages of the treatment plan based on the final position and initial position. For example, an intermediate stage may be a stage halfway between the initial positions and the final positions. The treatment plan may include an initial stage, based on the initial position of the teeth, a final stage, based on the final position of the teeth, and the one or more intermediate stages. In some embodiments, the downstream application 404 may employ automated quality control rules or algorithms to ensure that collisions do not occur at any stage, or any collisions are less than a certain intrusion depth (e.g., less than 0.5 mm). The automated quality control rules or algorithms may also ensure that certain teeth (such as centrals) are located at approximately a midline of the detention. The downstream application 404 may adjust the final position of the teeth and/or the stages of the treatment plan based on an outcome of the automated quality control rules (e.g., to ensure that collisions satisfy the automated quality control rules, to ensure that teeth are located at approximately their intended position, etc.).

An administrator or other user may view the 3D teeth representation and/or the treatment plan, in addition to other information relating to the treatment plan (e.g., tooth movements, tooth rotations and translations, clinical indicators, the duration of the treatment plan, the orthodontic appliance that is prescribed to achieve the final tooth position (e.g., aligners), and the recommended wear time of the appliance to affect the final tooth position). The information related to the treatment plan may be determined based on historic treatment plans. For example, clinical indicators, the duration of the treatment plan, the orthodontic appliance that is prescribed to achieve the final tooth position, and the recommended wear time of the appliance to affect the final tooth position may be determined from a historic treatment plan with a similar initial position and similar final position. Similarly, the information relating to the treatment plan may take into account biomechanical and biological parameters relating to tooth movement, such as the amount and volume of tissue or bone remodeling, the rate of remodeling or the relating rate of tooth movement.

The downstream application 404 may also be configured to manufacture an aligner or other piece of hardware (e.g., a retainer). The downstream application 404 may use a final position of the teeth after treatment and/or a treatment plan to fabricate an aligner. In some embodiments, before the aligner is fabricated, the treatment plan may be approved by a remote dentist/orthodontist. For example, a 3D printing system (or other casting equipment) may cast, etch, or otherwise generate physical models based on the parametric models of one or more stages of the treatment plan. A thermoforming system may thermoform a polymeric material to the physical models, and cut, trim or otherwise remove excess polymeric material from the physical models to fabricate dental aligners (or retainers). The dental aligners or retainers can be fabricated using any of the systems or processes described in U.S. patent application Ser. No. 16/047,694, titled “Dental Impression Kit and Methods Therefor,” filed Jul. 27, 2018, and U.S. patent application Ser. No. 16/188,570, titled “Systems and Methods for Thermoforming Dental Aligners,” filed Nov. 13, 2018, now U.S. Pat. No. 10,315,353, the contents of each of which are hereby incorporated by reference in their entirety. The retainer may function in a manner similar to the dental aligners but to maintain (rather than move) a position of the patient's teeth. In some embodiments, the user 120 using the user device 121 and/or an orthodontist/dentist (or other licensed medical professional) may approve of the fabricated dental aligners by inputting, to the user device or treatment planning computing system 110 respectively, an approval/acknowledgement message. In some embodiments, the mesh cleanup application receives representations of the user's mouth periodically during the treatment plan. Accordingly, the downstream application 404 may keep a record of the progress of the user's teeth as the teeth move to the final position of treatment.

The downstream application 404 may also be configured to guide a user in placing an order based on a generated treatment plan and/or proposed aligners/retainers for facilitating the treatment plan. An order may be a transaction that exchanges money from a user 120 for a product (e.g., an impression kit, dental aligners, retainers, etc.). In some embodiments, the downstream application 404 may communicate prompts to the user device 121 to guide the user 120 through a payment/order completion system. The prompts may include asking the user 120 for patient information (e.g., name, physical address, email address, phone number, credit card information) and product information (e.g., quantity of product, product name). Responsive to receiving a user 120 input (and in some cases a medical user input from the treatment planning computing system 110), the downstream application 404 may initiate manufacturing (or printing/fabricating) the product. The user 120 may approve/acknowledge the order by interacting with an “Order Now” or “Pay Now” button (or other graphical indicator) on a user interface generated using the downstream application 404, interacting with a slider on the user interface, interacting with an object on the user device, making certain gestures and/or communicating audibly. In response to receiving an approval/acknowledgement from the user 120, the downstream application 404 initiates the product order. The initiated product order is transmitted to other downstream applications 404 to initiate the fabrication of one or more products (e.g., dental aligners). The initiated product order may also be transmitted to the treatment planning computing system 110 to be stored/recorded and/or associated with a user profile.

Downstream application 404 may also monitor a position of one or more teeth of the user 120 by comparing an expected teeth position (e.g., a final position of the treatment plan or other intermediate position of the treatment plan) to a current position of one or more teeth. The downstream application 404 may monitor the user's teeth to determine whether the user's treatment is progressing as expected. The downstream application 404 may determine that the user's treatment is progressing as expected by determining that the current position of the user's teeth is within a range of an expected teeth position. For instance, the downstream application 404 may compare each tooth, or a portion of each tooth, in a current position (including rotational and translational positions) to the corresponding tooth, or portion of each tooth, in an expected position (including rotational and translational positions). The downstream application 404 may be configured to prompt the user 120 to capture a representation of the user's teeth (e.g., images) to determine a current position of the user's teeth. In some embodiments, the downstream application 404 may convert the captured 2D image of the user's teeth to a parametric model of the user's teeth.

Referring to FIG. 5B, a flow diagram 520 of the mesh cleanup application 125 of FIG. 1 is shown, accordingly to an illustrative embodiment. The operations performed by the mesh cleanup application 125 (including the reception circuit 135, mesh output circuit 137, and the mesh processing circuit 133) vary according to the received representation of the user mouth 402.

Steps 522, 524, and 526 depict how the mesh cleanup application 125 receives a 3D representation of a mesh and applies a transformation function f to convert this 3D mesh into a 2D image representation. This process might involve encoding specific 3D surface characteristics into channels of the 2D representation. For example, the encoding process might include the transfer of labels, which could represent features like texture, curvature, or material properties, from the vertices of the mesh to corresponding pixels in the 2D image. For example, the 2D representation produced in step 526 could include a multitude of channel dimensions, representing attributes such as normals, depth information, color values, or albedo.

In general, function f can be a transformation function that maps each vertex (or surface characteristic) in the original 3D mesh to a corresponding pixel in the 2D image representation (i.e., effectively flattening the 3D model into a 2D space). This mapping can include spatial positioning and various surface characteristics, encoding them into corresponding channels in the 2D image. Conversely, function g can be inverse of function f, which takes the enhanced 2D image and maps it back onto the 3D mesh, reapplying the enhanced surface characteristics from the 2D pixels to their corresponding vertices in the 3D space. For example, function f could be a UV mapping process, which projects the texture map onto the 3D model. In this process, every vertex in the 3D model can be assigned a pair of coordinates in the 2D texture map, known as UV coordinates. This transformation can position each vertex on the 2D plane and encodes various surface characteristics into the texture map. In another example, function f could be a spherical projection method. Each vertex on the 3D model can be projected onto a 2D spherical surface enveloping the model, with the mapping preserving the relative positions and surface details of the vertices.

Step 528 includes the mesh cleanup application 125 applying a trained machine learning model to the 2D representation of the user's mouth or other body parts. The machine learning model, leveraging its training data, can operate on the 2D representation to determine an enhanced version of the user's mouth in the 2D space. This could include tasks such as smoothing, hole-filling, texture enhancement, or artifact removal, among others.

In steps 530, 532, and 534, the mesh cleanup application 125 can apply a reverse transformation function g to convert the labeled (and sometimes enhanced) 2D representation back into a 3D mesh. This process may include decoding the surface characteristics from the labels assigned to pixels in the 2D representation. For example, the decoding process might include transferring the labels from the pixels in the 2D representation back to their corresponding vertices in the 3D mesh, thus translating the enhancements made in the 2D space to the 3D model.

Referring to FIG. 5C, a flow diagram 540 of the mesh cleanup application 125 of FIG. 1 is shown, accordingly to an illustrative embodiment. The operations performed by the mesh cleanup application 125 (including the reception circuit 135, mesh output circuit 137, and the mesh processing circuit 133) vary according to the received representation of the user mouth 402.

In steps 541, 543, and 545, the application 125 first receives a 3D mesh representation of the user's mouth at a specific time point A. A transformation function, f1, can then applied to this 3D mesh to generate a corresponding 2D image representation. For example, this transformation could involve processes like UV mapping or spherical projection to maintain spatial and surface details of the original 3D mesh in the 2D image. Parallel to this, steps 542, 544, and 546 follow a similar procedure for a different time point B. In some implementations, the mesh cleanup application 125 receives a 3D mesh at time point B and applies a potentially different transformation function, f2, to create another 2D image representation. In some implementations, the mesh cleanup application 125 can apply the same transformation function, f1, but at a different time point B.

Subsequently, in step 547, the application 125 can perform a variety of information aggregation and combination operations. This includes but is not limited to operations such as channel concatenation, channel averaging, or the application of specific (non) linear functions to the two sets of input channels. In some implementations, step 547 could include the use of a dedicated machine learning (ML) model that generates a myriad of channels. For example, one channel might represent the combination of edge sharpness from the depth channel, color contrast from the RGB channel, and curvature information from the surface normal map. While these channels might seem insignificant to human observers, they can be tuned by the ML model to extract and encapsulate information that improves the efficiency and performance of downstream models in tasks such as 3D reconstruction, movement prediction, or texture mapping. Additionally, or alternatively, in cases where the ML model may not be preferred or feasible, different image processing techniques can also be applied in step 547. For example, a combination of edge detection algorithms, color histogram comparisons, and curvature analysis could be used to create a composite channel that, like the ML model, integrates multiple aspects of the data into a single representation.

With reference to specific aggregation and combination operations, channel aggregation and combination operations can be implemented in various ways, depending on the nature of the data and the specific use case. For example, the ML model step 547 could merge (e.g., using various information aggregation and combination operations) the individual 2D representations generated from both time points A and B, creating a combined 2D representation that captures the temporal dynamics of the user's mouth. In another example, channel concatenation could include stacking the color channels (e.g., RGB or grayscale) of the 2D images from both time points, leading to a multi-layered 2D representation. In another example, channel concatenation could include combining other feature channels such as surface normal maps, curvature data, texture information, or even data from the depth channel if available. In yet another example, channel averaging could include computing the mean of corresponding pixels across the two time points for each color channel, resulting in a single 2D representation that captures the average color values between the two points in time. In yet another example, (non) linear functions may be performed which could include using a function like a sigmoid or a rectified linear unit (ReLU) on the depth channel data from both time points, to highlight specific depth ranges or suppress depth information that falls outside of certain thresholds. In some embodiments, dedicated machine learning models can be developed and trained specifically for the task of effectively combining different channels of data, such as color, depth, and surface normal information, from multiple time points. These dedicated models can learn to identify and emphasize the features in the aggregated data, thereby producing combined representations that are particularly suited to the downstream applications for which the 3D model is being generated.

In steps 548 and 549, the combined 2D representation can then be used as an input for a machine learning model. The model works to identify and annotate key features in the image, producing a labeled (and possibly enhanced) 2D representation. Lastly, if the 2D representation is improved or there's a need or desire to revert back to a 3D model, steps 550 and 552 include applying an inverse function, denoted as gf. For example, this function can represent the average inverse of f1 and f2, transforming the labeled 2D representation back into an enhanced and labeled 3D mesh representation. In another example, this function could employ weights, where different weights are assigned to the inverses of f1 and f2 based on certain criteria, such as the reliability of the data from each time point or the temporal distance to the target time point. In some implementations, after the execution of the machine learning model in step 549, the process could come to a conclusion at step 551, particularly if no further 3D reconstruction is required.

Referring to FIG. 5D, a flow diagram 560 of the mesh cleanup application 125 of FIG. 1 is shown, accordingly to an illustrative embodiment. The operations performed by the mesh cleanup application 125 (including the reception circuit 135, mesh output circuit 137, and the mesh processing circuit 133) vary according to the received representation of the user mouth 402.

Steps 561, 562, and 563 (similar to FIG. 5B) include applying function f to a 3D representation of a mesh to create a 2D representation of the mesh. This 2D representation can then be subjected to a sequence of specialized sub-problem machine learning models, as illustrated in steps 564A, 564B, and 564C. For instance, machine learning model sub-problem A at step 564A might be trained to focus on detecting and enhancing certain salient features, such as teeth boundaries or cavity details. Each sub-problem can output an enhanced 2D representation, as demonstrated in steps 565A, 565B, and 565C. These enhanced 2D representations, each reflecting one or more specific aspects of facets of the overall representation, serve as inputs to a main-problem machine learning model at step 566. In some implementations, the main model can aggregate or combine the multiple perspectives, generating a single, unified, and enhanced 2D representation. For example, at step 566, the mesh cleanup application 125 may be configured to combine the outcomes of each sub-problem, incorporating information from each to synthesize a complete detailed picture of the user's mouth in 2D.

Additionally, and similar to FIGS. 5B and 5C, an inverse function can be applied to this enhanced 2D representation at the final stage. Using the encoded surface characteristics, this function converts the detailed 2D image back into an enhanced 3D mesh representation for subsequent analysis or application. Furthermore, the 3D representation can be inputted into a machine learning model learner at step 568 and/or saved as machine learning model training data for future training by one or more machine learning models.

Referring to FIG. 5E, a flow diagram 580 of the mesh cleanup application 125 of FIG. 1 is shown, accordingly to an illustrative embodiment. The operations performed by the mesh cleanup application 125 (including the reception circuit 135, mesh output circuit 137, and the mesh processing circuit 133) vary according to the received representation of the user mouth 402.

Steps 581, 582, 583, 584A, 584B, 585A, 585B include similar actions and processes as described above with reference to FIG. 5D. Specifically, steps 584A and 584B involve the application of sub-problem A and B machine learning models, respectively, each producing an enhanced 2D representation (e.g., #1 and #2) as output at steps 585A and 585B. However, at step 586, an inverse function g (e.g., potentially the inverse of function f applied to the 3D mesh) is applied to one of these enhanced 2D representations (#2, for example). This action results in an enhanced 3D representation #2 at step 587. Following this, steps 588 and 589 involve running this enhanced 3D representation #2 through another machine learning model targeting sub-problem D, which subsequently outputs an enhanced 3D representation #3. As shown, interactive refinement can be used to enhance the quality and fidelity of the model.

Additionally, as shown a separate process (e.g., parallel or sequential) using machine learning model sub-problem C, at steps 592 and 593, can be used to output an enhanced 3D representation #1. That is, instead of using function f to create a 2D representation from the 3D representation, a machine learning model can be applied to the 3D representation. After applying machine learning model sub-problem C to output the enhanced 3D representation #1, at steps 594 and 595, the function f can be applied to create an enhanced 2D representation #3. That is, instead of enhancing the 2D representation, the 3D representation could be enhanced prior to applying function f.

In some implementations, each of the outputs, enhanced 2D representation #1, enhanced 3D representation #3, and enhanced 2D representation #3 can be inputted to machine learning model summary model at step 590. The combination process in step 590 could involve the use of a higher-level machine learning model that takes the diverse enhanced representations as its inputs. In some implementations, each input would pass through a dimensionality reduction step (e.g., via a method such as Principal Component Analysis (PCA) or an autoencoder). Subsequently, the reduced-dimensional inputs would be concatenated along a new dimension, forming a unified feature vector that serves as the final output of the summary model.

In some implementations, the combination could be carried out using a fusion technique, such as late fusion or early fusion, which either combines the outputs of each individual machine learning model or merges the inputs before being processed by the summary model, respectively. In some implementations, in the context of UV mapping, an enhanced 3D representation and an enhanced 2D representation could be combined by associating each vertex in the 3D representation with coordinates on the 2D image. This process would allow texture or other information (collectively surface characteristics) from the 2D representation to be mapped onto the 3D representation, blending both forms of data. At step 591, the process can end.

One of the advantages of this process is its flexibility in accepting various combinations of outputs at the summary stage. For example, the summary model can handle input sets ranging from three (or more) enhanced 2D representations, a mix of 2D and 3D representations, all 3D representations, and even combinations of enhanced and unenhanced representations. This adaptability allows the system to accommodate a wide array of use cases and input conditions.

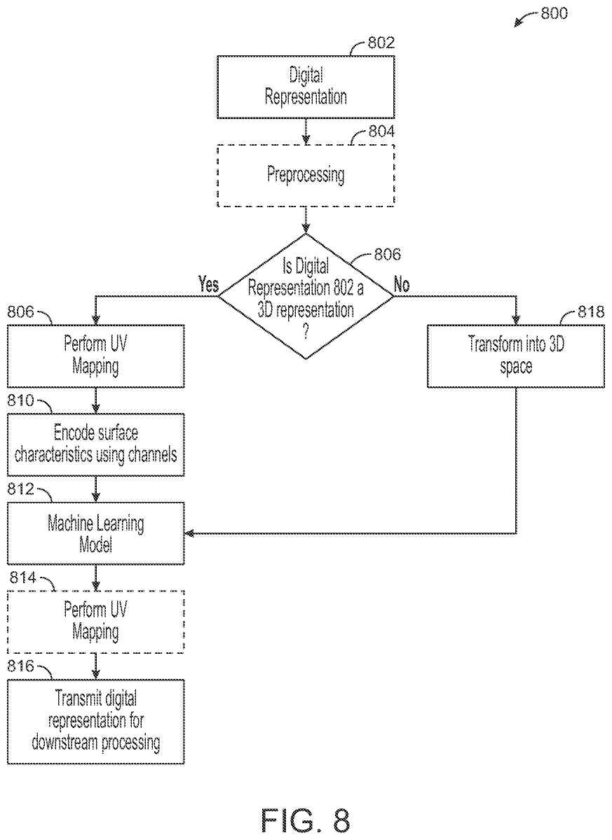

FIG. 8 is another flow diagram 800 of the mesh cleanup application of FIG. 1, according to an illustrative embodiment. The mesh cleanup application 125 may ingest one or more digital representations 802 for subsequent processing. The digital representation(s) 802 may be received from a user device 121 or other equipment (e.g., intraoral scanning device, dental impression kit). In some embodiments, the digital representation(s) may be 2D representations (e.g., images, frames of a video). In other embodiments, the digital representation(s) 802 may be 3D representations (e.g., a scan). For convenience, the digital representation(s) 802 will be referred to as a single digital representation 802.

In some implementations, the mesh cleanup application 125 executing the reception circuit 135 may perform one or more preprocessing operations 804 on digital representation 802. The preprocessing operation 804 may be configured to standardize and/or normalize the digital representation 802, scale the digital representation 802, extract features from the digital representation 802, perform pose estimation on the digital representation 802, and the like.

In some implementations, if preprocessing operation 804 is employed to preprocess the digital representation 802, a post-processing engine (not shown) may be employed to modify, correct, adjust, or otherwise process the output data. For example, if an image is converted to greyscale during preprocessing, then the image may be converted back to a color image during post-processing.

The mesh cleanup application 125 determines whether the digital representation 802 is a 3D representation in decision 806. The digital representation 802 may be 3D representation if the digital representation 802 is a CT image, a model from a dental impression, and the like.

If the digital representation 802 is a 3D representation, then the mesh cleanup application 125 executing the mesh processing circuit 133 maps the 3D digital representation 802 into a 2D image space. In some embodiments, the mesh processing circuit 133 employs UV mapping 806 such that the textures of the 3D representation are conserved (e.g., differentiating teeth from gums, differentiating teeth from other teeth). UV mapping unwraps the 3D representation 802, assigning pixels to the surface of the 3D representation by encoding orientation information associated with each face. As the mesh processing circuit 133 maps the digital representation from 3D space to 2D space, the mesh processing circuit 133 encodes the 3D surface characteristics (e.g., face or vertex normals, curvature, other dimensional representations) for each surface 810 (e.g., of a polygon). For example, each edge of a face may be encoded with one or more channels of the 2D representation (e.g., RGB values). In another example, each edge of a face may be encoded with one or more channels of the 2D representation. As described herein, encoding the element surface characteristics allows the mesh processing circuit 133 to track the orientation of each face in space. Accordingly, encoding the element surface characteristics allows the mesh processing circuit 133 to reconstruct the 3D digital representation from 2D space.

In some implementations, the mesh processing circuit 133 can define a function that maps each vertex in the 3D digital representation to a corresponding pixel in the 2D image space. This function facilitates translating the representation from a 3D space to a 2D space and facilitates communication of information between these two distinct representations. For example, encoding one or more 3D surface characteristics of the plurality of surfaces, such as the normals, using one or more channels of the 2D representation, like RGB values, can be interpreted as a means of transferring inherent attributes from the mesh vertices to their equivalent pixels in the 2D space. Conversely, decoding involves the transfer of generated labels in the 2D space, which may encompass one or more channels representing 3D surface characteristics, from their corresponding pixels back to their original vertices in the 3D mesh. This bidirectional transfer process provides a consistent representation and manipulation of the data across both 2D and 3D domains.

The mesh application 125 may utilize the mesh processing circuit 133 to apply a machine learning model 812. As described with reference to FIG. 3, the machine learning model is trained to clean/enhance the 2D representation. Accordingly, given a representation of the user's mouth, the machine learning model is trained to smooth, remove artifacts, fill holes, and the like. The enhanced representation of the user's mouth is a representation of the user's mouth that represent the user's true teeth and gums of which the digital representation 802 is based on. It should be appreciated that in some embodiments, the mesh cleanup application 125 proceeds directly to applying the machine learning model 812 to the digital representation 802 (with optional preprocessing 804). In these embodiments, the machine learning model 812 may be configured to receive 3D representations and trained using 3D input-output pairs. For example, the machine learning model 812 may be PointNet or PointNet++. Accordingly, the mesh cleanup application 125 does not need to reduce the dimension of the digital representation 802 by performing UV mapping 06.

Still referring to FIG. 8, if the mesh cleanup application 125 determines that the digital representation 802 is not a 3D representations (e.g., a 2D image), then the mesh cleanup application 125 (executing the reception circuit 135) may transform 818 the representation into one or more 3D representations. For example, the mesh cleanup application 125 may be configured to extract depth data from a 2D image (e.g., via the reception circuit 135 using photogrammetry, for instance.) In some embodiments, the reception circuit 135 may employ one or more mesh generation models to create a 3D mesh representation. The machine learning model 812 may be applied to the newly transformed 3D representation. For example, the machine learning model 812 may be PointNet or PointNet++. The machine learning model 812 predicts the enhanced 3D representation of the user's mouth.

If the machine learning model 812 predicts enhanced 2D representations of the user's mouth, then the mesh application 125 may utilize the mesh processing circuit 133 to perform UV mapping 814. The mesh processing circuit 133 performs UV mapping to convert the 2D enhanced representation of the user's mouth into a 3D enhanced representation of the user's mouth. The mesh application utilizes the encoded 3D surface characteristics to reconstruct the 3D representation using one or more channels of the 2D representation. In some implementations, to facilitate a mesh enhancement process, the mesh processing circuit 133 can define a function to map each vertex in the original 3D digital representation to a corresponding pixel in the 2D image space. This function establishes a correspondence between the 3D mesh and its 2D counterpart. After the machine learning model 812 is applied to enhance the 2D representation (e.g., smoothing, artifact removal, hole filling, etc.), the same mapping function can be used in reverse. This involves decoding the enhanced labels in the 2D representation and transferring them back to their corresponding vertices in the 3D space. Accordingly, the improved surface characteristics in the 2D domain, such as enhanced RGB values representing the normals, can be reapplied to the 3D digital representation.

The mesh application 125 may employ the mesh output circuit 137 to transform the enhanced 3D representation into a representation suitable for one or more downstream applications. Additionally, or alternatively, the mesh application 125 transmits the enhanced 3D representation to one or more downstream applications. Example downstream applications that may receive the enhanced representation of the user's mouth are described with reference to FIG. 4.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (e.g., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the systems, apparatuses, and methods shown in the various exemplary embodiments is illustrative only. Additionally, any element disclosed in one embodiment may be incorporated or utilized with any other embodiment disclosed herein. For example, any of the exemplary embodiments described in this application can be incorporated with any of the other exemplary embodiment described in the application. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A system comprising:
one or more processors and a non-transitory computer readable medium containing instructions that when executed by the one or more processors causes the one or more processors to perform operations comprising:
receiving a three-dimensional (3D) representation of a user's mouth, wherein the 3D representation is a mesh representation comprising a plurality of surfaces;
mapping the 3D representation of the user's mouth into a two-dimensional (2D) space;
encoding one or more 3D surface characteristics of the plurality of surfaces using one or more channels of a 2D representation;
determining an enhanced representation of the user's mouth in the 2D space by executing a machine learning model on the representation of the user's mouth in the 2D space;
mapping the enhanced representation of the user's mouth in the 2D space to an enhanced 3D representation of the user's mouth using the one or more 3D surface characteristics of the plurality of surfaces; and
outputting the enhanced 3D representation.

2. The system of claim 1, wherein mapping the 3D representation of the user's mouth into the 2D space comprises using UV mapping, and wherein the UV mapping comprises mapping at least one vertex of the 3D representation to at least one pixel in the 2D space.

3. The system of claim 2, wherein UV mapping comprises assigning a plurality of pixels to each surface of the plurality of surfaces.

4. The system of claim 1, wherein mapping the enhanced representation of the user's mouth in 2D space to the enhanced 3D representation of the user's mouth comprises using UV mapping.

5. The system of claim 1, wherein the machine learning model is trained using a set of training 3D representations and corresponding enhanced 3D representations, the set of training 3D representations and corresponding enhanced 3D representations are mapped into 2D space using UV mapping.

6. The system of claim 1, wherein the instructions executed by the one or more processors causes the one or more processors to perform operations comprising:
sampling, from a database, a training 3D representation of a user's mouth and a corresponding enhanced 3D representation according to a flag associated with at least one of the training 3D representation or the enhanced 3D representation; and
training the machine learning model using the sampled training 3D representation and the enhanced 3D representation.

7. The system of claim 6, wherein the flag indicates at least one of a threshold customer satisfaction score, a threshold number of mouth conditions, or a threshold score of a mouth condition.

8. The system of claim 1, wherein encoding the one or more 3D surface characteristics comprises encoding a normal of each surface of the plurality of surfaces using red green blue (RGB) values associated with orientation information of each surface.

9. The system of claim 1, wherein the enhanced 3D representation is an enhanced mesh representation of the user's mouth that has been at least one of sculpted, smoothed, filled in, or has had artifacts removed, and wherein the one or more channels of the 2D representation is at least one of a 3-channel 2D representation, a 4-channel 2D representation, a 5-channel 2D representation, a 10-channel 2D representation, or an n-channel 2D representation.

10. The system of claim 1, wherein the instructions executed by the one or more processors cause the one or more processors to perform operations comprising:
receiving the 2D representation of the user's mouth; and
converting the 2D representation into the 3D representation of the user's mouth.

11. The system of claim 10, wherein the 2D representation of the user's mouth is based on a video stream obtained by a user device associated with the user.

12. A method comprising:
receiving, by one or more processors, a three-dimensional (3D) representation of a user's mouth, wherein the 3D representation is a mesh representation comprising a plurality of surfaces;
mapping, by the one or more processors, the 3D representation of the user's mouth into a two-dimensional (2D) space;
encoding, by the one or more processors, one or more 3D surface characteristics of the plurality of surfaces using one or more channels of a 2D representation;
applying, by the one or more processors, a machine learning model to the representation of the user's mouth in 2D space, the machine learning model trained to enhance the representation of the user's mouth in 2D space; and
mapping, by the one or more processors, the enhanced representation of the user's mouth in 2D space to an enhanced representation of the user's mouth in 3D space using the encoded one or more 3D surface characteristics.

13. The method of claim 12, further comprising:
generating, by the one or more processors, a treatment plan based on the enhanced representation of the user's mouth in 3D space.

14. The method of claim 13, further comprising receiving, by the one or more processors, from a treatment planning computing device, validation of the treatment plan.

15. The method of claim 13, wherein encoding one or more 3D surface characteristics comprises encoding a the normal of each surface of the plurality of surfaces using red green blue (RGB) values associated with orientation information of each surface.

16. The method of claim 13, further comprising receiving, by the one or more processers, from a user device, an initiation of an order of a product based on the treatment plan.

17. The method of claim 13, wherein generating the treatment plan comprises generating, by the one or more processes, a plurality of intermediate 3D representations of the user's mouth showing a progression of a plurality of teeth from an initial position to a final position, wherein each of the plurality of intermediate 3D representations corresponds to a respective stage of the treatment plan.

18. The method of claim 13, further comprising manufacturing a dental aligner specific to the enhanced representation of the user's mouth in 3D space.

19. A method comprising:
receiving, by one or more processors, a two-dimensional (2D) representation of a user's mouth;
converting, by the one or more processors, the 2D representation into a three-dimensional (3D) representation of the user's mouth, wherein the 3D representation is a mesh representation comprising a plurality of surfaces;
mapping, by the one or more processors, the 3D representation of the user's mouth into a 2D space;
encoding, by the one or more processors, one or more 3D surface characteristics of the plurality of surfaces using one or more channels of the 2D representation;
applying, by the one or more processors, a machine learning model to the representation of the user's mouth in 2D space, the machine learning model trained to enhance the representation of the user's mouth in 2D space; and
mapping, by the one or more processors, the enhanced representation of the user's mouth in 2D space to an enhanced representation of the user's mouth in 3D space using the encoded one or more 3D surface characteristics.

20. The method of claim 19, wherein mapping, by the one or more processors, the 3D representation of the user's mouth into the 2D space and mapping, by the one or more processors, the enhanced representation of the user's mouth in 2D space to the enhanced representation of the user's mouth in 3D space comprises performing UV mapping, and wherein the UV mapping comprises mapping at least one vertex of the 3D representation to at least one pixel in the 2D space.
